(19) United States
(12) Patent Application Publication
(10) Pub. No.: US 2006/0292600 A1
(43) Pub. Date: Dec. 28, 2006

(54) Methods, compositions and kits for the detection and monitoring of lung cancer

(75) Inventors: Barbara K. Zehentner-Wilkinson, Poulsbo, WA (US); Dawn C.J. Hayes, Preston, WA (US); Raymond L. Houghton, Bothell, WA (US)

Correspondence Address: SEED INTELLECTUAL PROPERTY LAW GROUP PLLC, 701 FIFTH AVE, SUITE 5400, SEATTLE, WA, 98104, US

(73) Assignee: Corixa Corporation, Brentford (GB)

(21) Appl. No.: 11/392,479

(22) Filed: Mar. 29, 2006

Related U.S. Application Data
(63) Continuation-in-part of application No. 10/550,797.
(60) Provisional application No. 60/719,549, filed on Sep. 21, 2005.

(51) Int. Cl. C12Q 1/68; C12P 19/34

(52) U.S. Cl. 435/6; 435/91.2

(57) ABSTRACT

Compositions and methods for the diagnosis of lung cancer are disclosed. Such methods are useful to detect early tumors or provide adequate stage/grade information or tumor specificity. Compositions may comprise one or more lung tumor proteins, teins, immunogenic portions thereof, or polynucleotides that encode such portions. Such compositions may be used, for example, to improve lung cancer diagnosis and prognosis and potentially differentiate between NSCLC and SCLC.

### STATEMENT REGARDING SEQUENCE LISTING SUBMITTED ON CD-ROM

The Sequence Listing associated with this application is provided on CD-ROM in lieu of a paper copy, and is hereby incorporated by reference into the specification. Three CD-ROMs are provided, containing identical copies of the sequence listing: CD-ROM No. 1 is labeled COPY 1, contains the file 609c1.app.txt which is 44.8 KB and created on Mar. 29, 2006; CD-ROM No. 2 is labeled COPY 2, contains the file 609c1.app.txt which is 44.8 KB and created on Mar. 29, 2006; CD-ROM No. 3 is labeled CRF (Computer Readable Form), contains the file 609c1.app.txt which is 44.8 KB and created on Mar. 29, 2006.

### TECHNICAL FIELD OF THE INVENTION

The present invention relates generally to the field of cancer diagnostics. More specifically, the present invention relates to methods, compositions and kits for the detection of lung cancer in patients with different type, stage and grade of tumors that employ oligonucleotide hybridization and/or amplification to simultaneously detect two or more tissue-specific polynucleotides in a biological sample suspected of containing lung cancer cells.

### BACKGROUND OF THE INVENTION

1. Field of the Invention

Lung cancer remains a significant health problem throughout the world. The failure of conventional lung cancer treatment regimens can commonly be attributed, in part, to delayed disease diagnosis. Although significant advances have been made in the area of lung cancer diagnosis, there still remains a need for improved detection methodologies that permit early, reliable and sensitive determination of the presence of lung cancer cells.

2. Description of the Related Art

Lung cancer has the highest mortality rate of any of the cancers and is one of the most difficult to diagnose early. There are an estimated 1 million deaths annually worldwide for this disease. According to the American Cancer Society in 2002 alone there were an estimated 169,200 new cases diagnosed and ˜154,900 deaths. Typically lung cancers are classified into two major types: Non-Small Cell Lung Carcinomas (NSCLC) comprising squamous, adeno and large cell carcinomas and Small Cell Lung Carcinomas (SCLC). These groups represent ˜75% and 25% of all lung tumors respectively with adenocarcinoma and squamous cell carcinoma being the most prevalent forms of NSCLC with large cell carcinomas being ˜10%. Within the group of NSCLC, adenocarcinoma is currently the most prominent form of lung cancer in younger persons, women of all ages, lifetime nonsmokers and long-term former smokers. SCLC typically fall into two subtypes oat cell and intermediate cell. Less common tumors include carcinoid and mesotheliomas among others but these represent only a small percentage of all lung tumors. In almost all cases early diagnosis of NSCLC is elusive and most lung cancers have already metastasized by the time they are detected. Only 16.7% are localized on initial diagnosis. If tumors can be detected at a point where they are confined then the combination of chemotherapy and radiation has a possibility of success but overall the 5 year prognosis is very poor with only 10-15% survival rate. The picture with SCLC is even bleaker only 6% localized at initial diagnosis and with 5 year survival rates of −6%.

X-ray and computer tomography of the chest and abdomen are frequently used in diagnosis of lung tumors but lack sensitivity for detecting small foci and usually detect tumors that have already metastasized. Sputum cytology as a potential screening method in high-risk individuals has only been partially effective and often does not yield tumor type. To stage the disease CAT scan, MRI or bone scans are used to evaluate the spread of disease. Treatment for lung cancer is typically surgical, radiological or chemotherapy or combinations thereof, but usually with poor outcome due to the late diagnosis of disease.

The current tests for lung cancer lack either the clinical sensitivity to detect early tumors or provide inadequate stage/grade information or lack tumor specificity due to their originating from other tumor types or being present in benign lung disorders. There is therefore a need to develop specific tests that can improve lung cancer diagnosis and prognosis and potentially differentiate between NSCLC and SCLC. The present invention achieves these and other related objectives by providing methods that are useful for the identification of tissue-specific polynucleotides, in particular tumor-specific polynucleotides, as well as antibodies and methods, compositions and kits for the detection and monitoring of cancer cells in a patient afflicted with the disease.

### SUMMARY OF THE INVENTION

According to one aspect of the invention, methods are provided for detecting the presence of cancer cells in a biological sample comprising the steps of: detecting the level of expression in the biological sample of at least two cancer-associated markers selected from the group consisting of L762P, L550S, L587S and L984P; and, comparing the level of expression detected in the biological sample for each marker to a predetermined cut-off value for each marker; wherein a detected level of expression above the predetermined cut-off value for one or more markers is indicative of the presence of cancer cells in the biological sample.

The cancer to be detected according the methods of the invention may be any cancer type that expresses one or more of the cancer-associated markers described herein. In certain illustrative embodiments, the cancer is a lung cancer, pancreatic cancer, kidney cancer, bladder cancer or breast cancer. In a preferred embodiment, the cancer is a lung cancer, such as a small cell lung cancer or a non-small cell lung cancer.

The biological sample to be tested according to the methods of the invention may be any type of biological sample suspected of containing cancer-associated markers, antibodies to such cancer-associated markers and/or cancer cells expressing such markers or antibodies. In one embodiment, for example, the biological sample is a tissue sample suspected of containing cancer cells. In other embodiments, the biological sample is selected from the group consisting of a biopsy sample, lavage sample, sputum sample, serum sample, peripheral blood sample, lymph node sample, bone marrow sample, urine sample, and pleural effusion sample.

In certain embodiments of the invention, the step of detecting expression of a cancer-associated marker comprises detecting mRNA expression of a cancer-associated marker, for example, using a nucleic acid hybridization technique or a nucleic acid amplification method. Such methods for detecting mRNA expression are well-known and established in the art and may include, but are not limited to, transcription-mediated amplification (TMA), polymerase chain reaction amplification (PCR), ligase chain reaction amplification (LCR), strand displacement amplification (SDA), and nucleic acid sequence based amplification (NASBA), as further described herein. In certain preferred embodiments, the L762P mRNA comprises a nucleic acid sequence set forth in SEQ ID NO: 1 or a nucleic acid sequence encoding an amino acid sequence set forth in SEQ ID NO: 2, the L550S mRNA comprises a nucleic acid sequence set forth in SEQ ID NO: 5, the L587S mRNA comprises a nucleic acid sequence set forth in SEQ ID NO: 26 or a nucleic acid sequence encoding an amino acid sequence set forth in SEQ ID NO: 27, and/or the L984P mRNA comprises a nucleic acid sequence set forth in SEQ ID NO: 3 or 39 or a nucleic acid sequence encoding an amino acid sequence set forth in SEQ ID NO: 4 or 40.

In certain other embodiments of the invention, the step of detecting expression of a cancer-associated marker comprises detecting protein expression of a cancer-associated marker. Methods for detecting protein expression may include any of a variety of well-known and established techniques. For example, in certain embodiments, the step of detecting protein expression comprises detecting protein expression using an immunoassay, such as an enzyme-linked immunosorbent assay (ELISA), an immunohistochemical assay, an immunocytochemical assay, and/or a flow cytometry assay of antibody-labelled cells. In a preferred embodiment, the L762P protein comprises an amino acid sequence set forth in SEQ ID NO: 2, the L550S protein comprises the amino acid sequence set forth in SEQ ID NO:6, the L587S protein comprises an amino acid sequence sequence set forth in SEQ ID NO: 27, and/or the L984P protein comprises an amino acid sequence set forth in SEQ ID NO: 4 or 40.

In a more particular embodiment of the invention, a method is provided for detecting the presence of lung cancer cells in a biological sample comprising the steps of: detecting the level of mRNA expression in the biological sample of at least two cancer-associated markers selected from the group consisting of L762P, L550S, L587S and L984P, using a nucleic acid amplification method; and, comparing the level of expression detected in the biological sample for each marker to a predetermined cut-off value for each marker; wherein a detected level of expression above the predetermined cut-off value for one or more markers is indicative of the presence of lung cancer cells in the biological sample. In a preferred embodiment, the nucleic acid amplification method is selected from the group consisting of transcription-mediated amplification (TMA), polymerase chain reaction amplification (PCR), ligase chain reaction amplification (LCR), strand displacement amplification (SDA), and nucleic acid sequence based amplification (NASBA). In certain preferred embodiments, the L762P mRNA comprises a nucleic acid sequence set forth in SEQ ID NO: 1 or a nucleic acid sequence encoding an amino acid sequence set forth in SEQ ID NO: 2, the L550S mRNA comprises a nucleic acid sequence set forth in SEQ ID NO: 5, the L587S mRNA comprises a nucleic acid sequence set forth in SEQ ID NO: 26 or a nucleic acid sequence encoding an amino acid sequence set forth in SEQ ID NO: 27, and/or the L984P mRNA comprises a nucleic acid sequence set forth in SEQ ID NO: 3 or 39 or a nucleic acid sequence encoding an amino acid sequence set forth in SEQ ID NO: 4 or 40.

In another more particular embodiment of the invention, a method is provided for detecting the presence of lung cancer cells in a biological sample comprising the steps of: detecting the level of protein expression in the biological sample of at least two cancer-associated markers selected from the group consisting of L762P, L550S, L587S and L984P, using an immunoassay; and, comparing the level of expression detected in the biological sample for each marker to a predetermined cut-off value for each marker; wherein a detected level of expression above the predetermined cut-off value for one or more markers is indicative of the presence of lung cancer cells in the biological sample. In a preferred embodiment, the immunoassay is selected from the group consisting of an ELISA, an immunohistochemical assay, an immunocytochemical assay, and/or a flow cytometry assay of antibody-labelled cells. In certain preferred embodiments, the L762P protein comprises an amino acid sequence set forth in SEQ ID NO: 2, the L550S protein comprises an amino acid sequence set forth in SEQ ID NO:6, the L587S protein comprises an amino acid sequence set forth in SEQ ID NO: 27, and/or the L984P protein comprises an amino acid sequence set forth in SEQ ID NO: 4 or 40.

In another aspect, methods are provided for monitoring the progression of a cancer in a patient comprising the steps of: (a) detecting the level of expression in a biological sample from the patient of at least two cancer-associated markers selected from the group consisting of L762P, L550S, L587S and L984P; (b) repeating step (a) using a biological sample from the patient at a subsequent point in time; and, (c) comparing the level of expression detected in step (a) for each marker with the level of expression detected in step (b) for each marker. Using such an approach, a level of expression that is found to be increased at the subsequent point in time may be indicative of the presence of an increased number of cancer cells in the biological sample, which may be indicative of cancer progression in the patient from whom the biological sample was derived. Alternatively, a level of expression that is found to be decreased at the subsequent point in time may be indicative of the presence of fewer cancer cells in the biological sample, which may be indicative of a reduction of disease in the patient from whom the biological sample was derived.

In related aspects, methods are provided for monitoring the treatment of a cancer in a patient comprising the steps of: (a) detecting the level of expression in a biological sample from the patient of at least two cancer-associated markers selected from the group consisting of L762P, L550S, L587S and L984P; (b) repeating step (a) using a biological sample from the patient at a subsequent point in time; and, (c) comparing the level of expression detected in step (a) for each marker with the level of expression detected in step (b) for each marker. Using such an approach, a level of expression that is found to be increased at the subsequent point in time may be indicative of the presence of an increased number of cancer cells in the biological sample, which may be indicative of poor treatment responsiveness of the patient from whom the biological sample was derived. Alternatively, a level of expression that is found to be decreased at the subsequent point in time may be indicative of the presence of fewer cancer cells in the biological sample, which may be indicative of therapeutic responsiveness of the patient from whom the biological sample was derived.

The present invention further provides methods for detecting the presence of cancer cells in a biological sample comprising the steps of: contacting the biological sample with at least two polypeptides selected from the group consisting of L762P, L550S, L587S and L984P; and, detecting the presence of antibodies in the biological sample that are specific for one or more of the polypeptides; wherein the presence of antibodies specific for one or more of the polypeptides is indicative of the presence of cancer cells in the biological sample. Methods for detecting the presence of antibodies specific for a given polypeptide may include any of a variety of well-known and established techniques, illustrative examples of which are described herein.

According to another aspect of the invention, compositions and kits are provided for detecting cancer cells in a biological sample comprising at least two of: a first oligonucleotide specific for L762P; a second oligonucleotide specific for L550S; a third oligonucleotide specific for L587S; and, a fourth oligonucleotide specific for L984P. For example, in one illustrative embodiment, a composition according to this aspect of the invention may comprise at least two of the first, second, third and/or fourth oligonucleotides discussed above present together in the solid phase or in solution for use in a method of the present invention. In another illustrative embodiment, a kit according to this aspect of the invention may comprise at least two of the first, second, third and/or fourth oligonucleotides discussed above contained as separate components for use in a method of the present invention. In certain embodiments, the first oligonucleotide is specific for an L762P nucleic acid sequence set forth in SEQ ID NO: 1 or a nucleic acid sequence encoding an amino acid sequence set forth in SEQ ID NO: 2, the second oligonucleotide is specific for an L550S nucleic acid sequence set forth in SEQ ID NO: 5, the third oligonucleotide is specific for an L587S nucleic acid sequence set forth in SEQ ID NO: 26 or a nucleic acid sequence encoding an amino acid sequence set forth in SEQ ID NO:27, and/or the fourth oligonucleotide is specific for an L984P nucleic acid sequence set forth in SEQ ID NO: 3 or 39 or a nucleic acid sequence encoding an amino acid sequence set forth in SEQ ID NO: 4 or 40.

In a related aspect, compositions and kits are provided for detecting cancer cells in a biological sample comprising at least two of: a first primer pair specific for L762P; a second primer pair specific for L550S; a third primer pair specific for L587S; and, a fourth primer pair specific for L984P. For example, in one illustrative embodiment, a composition according to this aspect of the invention may comprise at least two of the first, second, third and/or fourth primer pairs discussed above present together in the solid phase or in solution for use in a method of the present invention. In another illustrative embodiment, a kit according to this aspect of the invention may comprise at least two of the first, second, third and/or fourth primer pairs discussed above contained as separate components for use in a method of the present invention. In certain preferred embodiments, the first, second, third and fourth primer pairs are effective in a nucleic acid amplification method for amplifying all or a portion of an L762P nucleic acid sequence set forth in SEQ ID NO: 1 or a nucleic acid sequence encoding an amino acid sequence set forth in SEQ ID NO: 2, an L550S nucleic acid sequence set forth in SEQ ID NO: 5, an L587S nucleic acid sequence set forth in SEQ ID NO: 26 or a nucleic acid sequence encoding an amino acid sequence set forth in SEQ ID NO: 27, and/or an L984P nucleic acid sequence set forth in SEQ ID NO: 3 or 39 or a nucleic acid sequence encoding an amino acid sequence set forth in SEQ ID NO: 4 or 40, respectively.

In another related aspect, compositions and kits are provided for detecting cancer cells in a biological sample comprising at least two of: a first antibody specific for an L762P protein; a second antibody specific for an L550S protein; a third antibody specific for an L587S protein; and, a fourth antibody specific for an L984P protein. For example, in one illustrative embodiment, a composition according to this aspect of the invention may comprise at least two of the first, second, third and/or fourth antibodies discussed above present together in the solid phase or in solution for use in a method of the present invention. In another illustrative embodiment, a kit according to this aspect of the invention may comprise at least two of the first, second, third and/or fourth antibodies discussed above contained as separate components for use in a method of the present invention. In certain preferred embodiments, the L762P protein comprises an amino acid sequence set forth in SEQ ID NO: 2, the L550S protein comprises an amino acid sequence set forth in SEQ ID NO:6, the L587S protein comprises an amino acid sequence set forth in SEQ ID NO: 27, and the L984P protein comprises an amino acid sequence set forth in SEQ ID NO:4 or 40.

According to yet another aspect of the invention, an array is provided comprising at least two of: a first oligonucleotide specific for L762P; a second oligonucleotide specific for L550S; a third oligonucleotide specific for L587S; and, a fourth oligonucleotide specific for L984P. In a preferred embodiment, the first oligonucleotide is specific for an L762P nucleic acid sequence set forth in SEQ ID NO: 1 or a nucleic acid sequence encoding an amino acid sequence set forth in SEQ ID NO: 2, the second oligonucleotide is specific for an L550S nucleic acid sequence set forth in SEQ ID NO:5, the third oligonucleotide is specific for an L587S nucleic acid sequence set forth in SEQ ID NO: 26 or a nucleic acid sequence encoding an amino acid sequence set forth in SEQ ID NO: 27, and the fourth oligonucleotide is specific for an L984P nucleic acid sequence set forth in SEQ ID NO: 3 or 39 or a nucleic acid sequence encoding an amino acid sequence set forth in SEQ ID NO: 4 or 40.

In a related aspect, an array is provided comprising at least two of: a first antibody specific for an L762P protein; a second antibody specific for an L550S protein; a third antibody specific for an L587S protein; and, a fourth antibody specific for an L984P protein. In another related aspect, an array is provided comprising at least two of: an L762P protein or portion thereof; an L550S protein or portion thereof; an L587S protein or portion thereof; and, an L984P protein or portion thereof. In preferred embodiments, the L762P protein comprises an amino acid sequence set forth in SEQ ID NO: 2, the L550S protein comprises an amino acid sequence set forth in SEQ ID NO:6, the L587S protein comprises an amino acid sequence set forth in SEQ ID NO: 27, and the L984P protein comprises an amino acid sequence set forth in SEQ ID NO: 4 or 40.

The present invention provides methods for detecting the presence of lung cancer cells in a patient. In certain embodiments, the methods comprise the steps of: (a) obtaining a biological sample from the patient; (b) contacting the biological sample with two or more oligonucleotide pairs specific for independent polynucleotide sequences which are unrelated to one another, wherein the oligonucleotide pairs hybridize, under moderately stringent conditions, to their respective polynucleotides and the complements thereof (c) amplifying the polynucleotides; and (d) detecting the amplified polynucleotides; wherein the presence of one or more of the amplified polynucleotides indicates the presence of lung cancer cells in the patient.

By some embodiments, detection of the amplified polynucleotides may be preceded by a fractionation step such as, for example, gel electrophoresis. Alternatively or additionally, detection of the amplified polynucleotides may be achieved by hybridization of a labeled oligonucleotide probe that hybridizes specifically, under moderately stringent conditions, to such polynucleotides. Oligonucleotide labeling may be achieved by incorporating a radiolabeled nucleotide or by incorporating a fluorescent label.

In certain preferred embodiments, cells of a specific tissue type may be enriched from the biological sample prior to the steps of detection. Enrichment may be achieved by a methodology selected from the group consisting of cell capture and cell depletion. Exemplary cell capture methods include immunocapture and comprise the steps of: (a) adsorbing an antibody to a tissue-specific cell surface to cells said biological sample; (b) separating the antibody adsorbed tissue-specific cells from the remainder of the biological sample. Exemplary cell depletion may be achieved by cross-linking red cells and white cells followed by a subsequent fractionation step to remove the cross-linked cells.

Alternative embodiments of the present invention provide methods for determining the presence or absence of lung cancer in a patient, comprising the steps of: (a) contacting a biological sample obtained from the patient with two or more oligonucleotides that hybridize to two or more polynucleotides that encode two or more lung tumor proteins; (b) detecting in the sample a level of at least one of the polynucleotides (such as, for example, mRNA) that hybridize to the oligonucleotides; and (c) comparing the level of polynucleotides that hybridize to the oligonucleotides with a predetermined cut-off value, and therefrom determining the presence or absence of lung cancer in the patient. Within certain embodiments, the amount of mRNA is detected via polymerase chain reaction using, for example, at least one oligonucleotide primer that hybridizes to a polynucleotide encoding a polypeptide as recited above, or a complement of such a polynucleotide. Within other embodiments, the amount of mRNA is detected using a hybridization technique, employing an oligonucleotide probe that hybridizes to a polynucleotide that encodes a polypeptide as recited above, or a complement of such a polynucleotide.

In related aspects, methods are provided for monitoring the progression of lung cancer in a patient, comprising the steps of: (a) contacting a biological sample obtained from a patient with two or more oligonucleotides that hybridize to two or more polynucleotides that encode lung tumor proteins; (b) detecting in the sample an amount of the polynucleotides that hybridize to the oligonucleotides; (c) repeating steps (a) and (b) using a biological sample obtained from the patient at a subsequent point in time; and (d) comparing the amount of polynucleotide detected in step (c) with the amount detected in step (b) and therefrom monitoring the progression of the cancer in the patient.

Certain embodiments of the present invention provide that the step of amplifying said first polynucleotide and said second polynucleotide is achieved by the polymerase chain reaction (PCR).

The present invention also provides kits that are suitable for performing the detection methods of the present invention. Exemplary kits comprise oligonucleotide primer pairs each one of which specifically hybridizes to a distinct polynucleotide. Within certain embodiments, kits according to the present invention may also comprise a nucleic acid polymerase and suitable buffer.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

FIG. 1 shows illustrative sequence identification based on amplicon size. Multigene RT-PCR samples were separated by agarose gel electrophoresis (4%). Lane 2 shows the distinguishable amplicon sizes for multigene products L762P (249 bp), L550S (204 bp), L587S (169 bp), and L984P (157 bp) as indicated by ladder (lane 1) and labeled arrows. Lane 3 shows the no template, negative control.

FIG. 2 shows illustrative multigene RT-PCR analysis of 160 normal human tissue samples. 85 samples showed little reactivity, less than 92 combined gene copies/actin. PCR results were slightly elevated in normal skin, esophagus, trachea, and bronchus samples (n=9), less than 146 combined gene copies/actin. Normal lung PCR results were less than 7 combined gene copies/actin.

FIG. 3 shows illustrative multigene RT-PCR analysis of 108 blood samples from lung cancer patients with various types of disease (88 NSCLC and 20 SCLC) and 25 blood samples from healthy donors. Blood samples from 7/15 patients with no current evidence of disease (NED) receiving treatment, 5/12 patients with no current evidence of disease not receiving treatment, 27/64 patients with active disease receiving treatment, and 10/17 patients with active disease not receiving treatment showed signals above the cut-off value (2SD+mean normals=27.00; indicated by bold line).

FIGS. 4A-C show illustrative multigene RT-PCR analysis of repeat draws from Patient A with large cell lung carcinoma (FIG. 4A), Patient B with small cell lung carcinoma (FIG. 4B), and Patient C with squamous cell carcinoma (FIG. 4C). Arrow in FIG. 4B indicates clinical diagnosis of Patient B with progressive disease. Copies are low during effective chemotherapy, but signal increases at discontinuation of treatment prior to relapse.

### BRIEF DESCRIPTION OF SEQUENCE IDENTIFIERS

SEQ ID NO: 1 is the determined cDNA sequence L762P.

SEQ ID NO: 2 is the amino acid sequence encoded by the sequence of SEQ ID NO: 1.

SEQ ID NO: 3 is the determined cDNA sequence L984P.

SEQ ID NO: 4 is the amino acid sequence encoded by the sequence of SEQ ID NO: 3.

SEQ ID NO: 5 is the determined cDNA sequence L550S.

SEQ ID NO: 6 is the amino acid sequence encoded by the sequence of SEQ ID NO: 5.

SEQ ID NO: 7 is the determined cDNA sequence L552S.

SEQ ID NO: 8 is the amino acid sequence encoded by the sequence of SEQ ID NO: 7.

SEQ ID NO:9 is the DNA sequence of L552S INT forward primer.

SEQ ID NO:10 is the DNA sequence of L552S INT reverse primer.

SEQ ID NO:11 is the DNA sequence of L552S Taqman probe.

SEQ ID NO:12 is the DNA sequence of L550S INT forward primer.

SEQ ID NO:13 is the DNA sequence of L550S INT reverse primer.

SEQ ID NO:14 is the DNA sequence of L550S Taqman probe.

SEQ ID NO:15 is the DNA sequence of L726P INT forward primer.

SEQ ID NO:16 is the DNA sequence of L726P INT reverse primer.

SEQ ID NO:17 is the DNA sequence of L726P Taqman probe.

SEQ ID NO:18 is the DNA sequence of L984P INT forward primer.

SEQ ID NO:19 is the DNA sequence of L984P INT reverse primer.

SEQ ID NO:20 is the DNA sequence of L984P Taqman probe.

SEQ ID NO:21 is the determined cDNA sequence of L763P.

SEQ ID NO:22 is the amino acid sequence encoded by the sequence of SEQ ID NO:21.

SEQ ID NO:23 is the DNA sequence of L763P INT forward primer.

SEQ ID NO:24 is the DNA sequence of L763P reverse primer.

SEQ ID NO:25 is the DNA sequence of L763P Taqman probe.

SEQ ID NO:26 is the determined cDNA sequence of L587.

SEQ ID NO:27 is the amino acid sequence encoded by the sequence of SEQ ID NO:26.

SEQ ID NO:28 is the DNA sequence of L587 INT forward primer.

SEQ ID NO:29 is the DNA sequence of L587 INT reverse primer.

SEQ ID NO:30 is the DNA sequence of L587 Taqman probe.

SEQ ID NO:31 is the determined cDNA sequence of L523.

SEQ ID NO:32 is the amino acid sequence encoded by the sequence of SEQ ID NO:31.

SEQ ID NO:33 is the DNA sequence of L523 primer.

SEQ ID NO:34 is the DNA sequence of L523 primer.

SEQ ID NO:35 is the DNA sequence of another L550S INT reverse primer having the sequence 5′-TCGACTTATAGTCAGCMCATCCTTCT-3′.

SEQ ID NO:36 is an illustrative primer ActinF having the sequence 5′-ACTGGAACGGTGAAGGTGACA.

SEQ ID NO:37 is an illustrative primer ActinR having the sequence 5′-CGGCCACATTGTGAACTTTG.

SEQ ID NO:38 is an illustrative 6-carboxy-fluorescein (FAM)-labeled actin-specific probe having the sequence 5′-6FAM-CAGTCGGTTGGAGCGAGCATCCC-3′-TAMRA.

SEQ ID NO: 39 is an extended cDNA sequence encoding the cancer-associated marker L984S.

SEQ ID NO: 40 is the amino acid sequence encoded by the sequence of SEQ ID NO: 39. This sequence differs from SEQ ID NO:4 by 2 additional glutamine residues just before the alanine at position 63.

### DETAILED DESCRIPTION OF THE INVENTION

As noted above, the present invention is directed generally to methods that are suitable for the identification of tissue-specific polynucleotides as well as to methods, compositions and kits that are suitable for the diagnosis and monitoring of lung cancer, in particular such methods, compositions and kits are suitable for use in the diagnosis, differentiation and/or prognosis of NSCLC and SCLC. Such diagnostic methods will form the basis for a molecular diagnostic test for detecting lung cancer metastases in lung tissue and for the detection of anchorage independent lung cancer cells in blood as well as in mediastinal lymph nodes of distant metastases.

A variety of genes have been identified as over-expressed in lung tumors, in particular squamous or adeno forms of NSCLC or small cell carcinomas. These include, but are not limited to: L762P, L984P, L550S/L548S, L552S/L547S, L552/L547S, L200T, L514S, L551S, L587S, L763S, L773P, L801P. L985P, L1058C, L1081C, L523S, OF1783P, B307D (WIPO International Patent Application Nos: WO 99/47674, published Sep. 23, 1999; WO 00/61612, published Oct. 19, 2000; WO 02/00174, published Jan. 3, 2002; WO 02/47534, published Jun. 20, 2002; WO 01/72295, published Oct. 4, 2001; WO 02/092001, published Nov. 21, 2002; WO 01/00828, published Jan. 1, 2001; WO 02/04514, published Jan. 17, 2002; WO 01/92525, published Dec. 6, 2002; WO 02/02623, published Jan. 10, 2002. US patent Nos: Wang et al., U.S. Pat. No. 6,482,597, issued Nov. 22, 2002; Wang et al., U.S. Pat. No. 6,518,256, issued Feb. 11, 2003; Wang et al., U.S. Pat. No. 6,426,072, issued Jul. 30, 2002; Reed et al., U.S. Pat. No. 6,210,883, issued Apr. 3, 2001; Wang et al., U.S. Pat. No. 6,504,010, issued Jan. 7, 2003; Wang et al., U.S. Pat. No. 6,509,448, issued Jan. 21, 2003. Wang et al; Oncogene; 21(49):7598-604, 2002 (collagen type XI alpha 1).).

These genes were identified and characterized using PCR and cDNA library subtractions as well as electronic subtractions with each of the tumor types individually. The cDNAs identified were then evaluated by microarray then by Real Time PCR on tissue panels to identify specific expression patterns. Table 1 highlights the specificity of these genes for either adeno or squamous forms of NSCLC or both as well as genes specific for small cell lung carcinomas. In some cases reactivity with large cell carcinomas has also been identified by Real Time PCR analysis.

TABLE 1

| | | | | | Normal |
|---|---|---|---|---|---|
| Gene | Squamous | Adeno | Small cell | Large cell | Lung |
| L762P | +++++ | + | | | − |
| L984P | | + | +++ | | − |
| L550S/L548S | | +++++ | + | | − |
| L552S/L547S | ++ | +++++ | | | − |
| L200T | + | ++ | | ++ | − |
| L514S | ++++ | ++++ | | | − |
| L551S | | ++++ | | +/− | − |
| L587S | + | + | +++ | + | − |
| L763P | +++++ | | | | − |
| L773P | +++ | +++ | | | − |
| L801P | ++++ | ++++ | | ++ | − |
| L978P | + | ++ | +++++ | +/− | − |
| L985P | | + | +++++ | | − |
| L1058C | | | ++ | | − |
| L1081C | | | ++ | | − |
| L523S | +++++ | +++++ | + | ++ | − |
| OF 1783P | | | +++++ | | − |
| B307D | ++ | ++ | | + | − |

A first cancer-associated marker employed in the methods and compositions described herein and referred to as L762P belongs to the CLCA family of genes encoding calcium activated chloride channels (see, e.g., WO99/47674, incorporated herein by reference). This marker was initially identified in human lung, trachea, and mammary gland as hCLCA2. It has since been reported to be expressed in some squamous cell lung tumors (Gruber et al., Am J Physiol. 276: C1261-70, 1999; Konopitzky et al., J Immunol. 169: 540-7, 2002). A second cancer-associated marker employed in the methods and compositions of the present invention and referred to as L550S is a gene encoding the human high mobility group protein 2a (HMG2a), a family member of high mobility group architectural genes restricted to embryogenesis and normally diminished or silent in adult tissues (see, e.g., WO01/00828, incorporated herein by reference; Tessari et al., Mol Cell Biol. 23: 9104-16, 2003; Pentimalli et al., Cancer Res. 63: 7423-7, 2003). A third cancer-associated marker employed in the methods and compositions described herein and referred to as L587S is a gene located on chromosome 18 (see, e.g., WO02/02623, incorporated herein by reference). Subtraction libraries indicate that this marker has some association with cancer (Bangur et al., Oncogene. 21: 3814-25, 2002; Wang et al., Oncogene. 19: 1519-28, 2000). A fourth cancer-associated marker employed in the methods and compositions of the present invention and referred to L984P is a gene sharing homology with a gene encoding the Aschaetescute homologous protein ASH-1 (HASH1, ASCL1), which is expressed in some lung tumors (see, e.g., WO02/04514, incorporated herein by reference; Bangur et al., Oncogene. 21: 3814-25, 2002). Although certain of these markers have been individually shown to have some association with cancer, the discovery that their use in specific combination can offer the very high degree of complementation and tumor coverage demonstrated herein, particularly for lung cancer, was unexpected.

Identification of Tissue-Specific Polynucleotides

Certain embodiments of the present invention provide methods, compositions and kits for the detection of lung cancer cells within a biological sample from patients with different type, stage and grade of tumors. These methods comprise the step of detecting one or more tissue-specific polynucleotide(s) from a patient's biological sample the over-expression of which polynucleotides indicates the presence of lung cancer cells within the patient's biological sample. Accordingly, the present invention also provides methods that are suitable for the identification of tissue-specific polynucleotides. As used herein, the phrases “tissue-specific polynucleotides” or “tumor-specific polynucleotides” are meant to include all polynucleotides that are at least two-fold over-expressed as compared to one or more control tissues. As discussed in further detail herein below, over-expression of a given polynucleotide may be assessed by a variety of detection methods, including but not limited to microarray and/or quantitative real-time polymerase chain reaction (Real-time PCR™) methodologies.

Exemplary methods for detecting tissue-specific polynucleotides may comprise the steps of: (a) performing a genetic subtraction to identify a pool of polynucleotides from a tissue of interest; (b) performing a DNA microarray analysis to identify a first subset of said pool of polynucleotides of interest wherein each member polynucleotide of said first subset is at least two-fold over-expressed in said tissue of interest as compared to a control tissue; and (c) performing a quantitative polymerase chain reaction analysis on polynucleotides within said first subset to identify a second subset of polynucleotides that are at least two-fold over-expressed as compared to said control tissue.

Polynucleotides Generally

As used herein, the terms “polynucleotide” “nucleotide sequence” and “nucleic acid sequence” are used interchangeably and refer generally to either DNA or RNA molecules. Polynucleotides may be naturally occurring as normally found in a biological sample such as blood, serum, lymph node, bone marrow, sputum, urine and tumor biopsy samples. Alternatively, polynucleotides may be derived synthetically by, for example, a nucleic acid polymerization reaction. As will be recognized by the skilled artisan, polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e. an endogenous sequence that encodes a tumor protein, such as a lung tumor protein, or a portion thereof or may comprise a variant, or a biological or antigenic functional equivalent of such a sequence. Polynucleotide variants may contain one or more substitutions, additions, deletions and/or insertions, as further described below. The term “variants” also encompasses homologous genes of xenogenic origin.

When comparing polynucleotide or polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=4 and a comparison of both strands.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity.

Therefore, the present invention encompasses polynucleotide and polypeptide sequences having substantial identity to the sequences disclosed herein, for example those comprising at least 50% sequence identity, preferably at least 55%, 60%, 65%, 70%, 75%, 80%, 85%, 90%, 95%, 96%, 97%, 98%, or 99% or higher, sequence identity compared to a polynucleotide or polypeptide sequence of this invention using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

In additional embodiments, the present invention provides isolated polynucleotides and polypeptides comprising various lengths of contiguous stretches of sequence identical to or complementary to one or more of the sequences disclosed herein. For example, polynucleotides are provided by this invention that comprise at least about 15, 20, 30, 40, 50, 75, 100, 150, 200, 300, 400, 500 or 1000 or more contiguous nucleotides of one or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like.

The polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative DNA segments with total lengths of about 10,000, about 5000, about 3000, about 2,000, about 1,000, about 500, about 200, about 100, about 50 base pairs in length, and the like, (including all intermediate lengths) are contemplated to be useful in many implementations of this invention.

In other embodiments, the present invention is directed to polynucleotides that are capable of hybridizing under moderately stringent conditions to a polynucleotide sequence provided herein, or a fragment thereof, or a complementary sequence thereof. Hybridization techniques are well known in the art of molecular biology. For purposes of illustration, suitable moderately stringent conditions for testing the hybridization of a polynucleotide of this invention with other polynucleotides include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

Moreover, it will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Microarray Analyses

Polynucleotides that are suitable for detection according to the methods of the present invention may be identified, as described in more detail below, by screening a microarray of cDNAs for tissue and/or tumor-associated expression (e.g., expression that is at least two-fold greater in a tumor than in normal tissue, as determined using a representative assay provided herein). Such screens may be performed, for example, using a Synteni microarray (Palo Alto, Calif.) according to the manufacturer's instructions (and essentially as described by Schena et al., Proc. Natl. Acad. Sci. USA 93:10614-10619 (1996) and Heller et al., Proc. Natl. Acad. Sci. USA 94:2150-2155 (1997)).

Microarray is an effective method for evaluating large numbers of genes but due to its limited sensitivity it may not accurately determine the absolute tissue distribution of low abundance genes or may underestimate the degree of overexpression of more abundant genes due to signal saturation. For those genes showing overexpression by microarray expression profiling, further analysis was performed using quantitative RT-PCR based on Taqman™ probe detection, which comprises a greater dynamic range of sensitivity. Several different panels of normal and tumor tissues, distant metastases and cell lines were used for this purpose.

Quantitative Real-Time Polymerase Chain Reaction

Suitable polynucleotides according to the present invention may be further characterized or, alternatively, originally identified by employing a quantitative PCR methodology such as, for example, the Real-time PCR methodology. By this methodology, tissue and/or tumor samples, such as, e.g., metastatic tumor samples, may be tested along side the corresponding normal tissue sample and/or a panel of unrelated normal tissue samples.

Real-time PCR (see Gibson et al., Genome Research 6:995-1001, 1996; Heid et al., Genome Research 6:986-994, 1996) is a technique that evaluates the level of PCR product accumulation during amplification. This technique permits quantitative evaluation of mRNA levels in multiple samples. Briefly, mRNA is extracted from tumor and normal tissue and cDNA is prepared using standard techniques.

Real-time PCR may, for example, be performed either on the ABI 7700 Prism or on a GeneAmp® 5700 sequence detection system (Applied Biosystems, Foster City, Calif.). The 7700 system uses a forward and a reverse primer in combination with a specific probe with a 5′ fluorescent reporter dye at one end and a 3′ quencher dye at the other end (Taqman™). When the Real-time PCR is performed using Taq DNA polymerase with 5′-3′ nuclease activity, the probe is cleaved and begins to fluoresce allowing the reaction to be monitored by the increase in fluorescence (Real-time). The 5700 system uses SYBR® green, a fluorescent dye, that only binds to double stranded DNA, and the same forward and reverse primers as the 7700 instrument. Matching primers and fluorescent probes may be designed according to the primer express program (Applied Biosystems, Foster City, Calif.). Optimal concentrations of primers and probes are initially determined by those of ordinary skill in the art. Control (e.g., β-actin) primers and probes may be obtained commercially from, for example, Perkin Elmer/Applied Biosystems (Foster City, Calif.).

To quantitate the amount of specific RNA in a sample, a standard curve is generated using a plasmid containing the gene of interest. Standard curves are generated using the Ct values determined in the real-time PCR, which are related to the initial cDNA concentration used in the assay. Standard dilutions ranging from 10-10⁶ copies of the gene of interest are generally sufficient. In addition, a standard curve is generated for the control sequence. This permits standardization of initial RNA content of a tissue sample to the amount of control for comparison purposes.

In accordance with the above, and as described further below, the present invention provides the illustrative lung tissue- and/or tumor-specific polynucleotides L552S, L550S, L762P, L984P, L763P and L587 having sequences set forth in SEQ ID NOs: 1, 3, 5, 7, 21 and 26, illustrative polypeptides encoded thereby having amino acid sequences set forth in SEQ ID NO: 2, 4, 6, 8, 22 and 27 that may be suitably employed in the detection of cancer, more specifically, lung cancer.

Methodologies for the Detection of Cancer

In general, a cancer cell may be detected in a patient based on the presence of one or more polynucleotides, or the polypeptides encoded thereby, within cells of a biological sample (for example, blood, lymph nodes, bone marrow, sera, sputum, urine and/or tumor biopsies) obtained from the patient. In other words, such polynucleotides and polypeptides may be used as markers to indicate the presence or absence of a cancer such as, e.g., lung cancer. Further, cancer may be detected in a patient based on the presence of antibodies specific for the polypeptides.

Thus, in certain embodiments, the methods of the invention detect the expression of L762P, L550S, L587S and/or L984P mRNA in biological samples. Expression of the cancer-associated sequences of the invention may be detected at the mRNA level using methodologies well-known and established in the art, including, for example, in situ hybridization and/or any of a variety of nucleic acid amplification methods, as further described below.

Alternatively, or additionally, the methods described herein can detect the expression of L762P, L550S, L587S and/or L984P polypeptides in a biological sample using methodologies well-known and established in the art, including, for example, ELISA, immunohistochemistry, immunocytochemistry, flow cytometry and/or other known immunoassays, as further described below.

The cancer-associated sequences of the invention may be used in the detection of essentially any cancer type that expresses one or more such sequences, including lung cancer, pancreatic cancer, kidney cancer, bladder cancer, breast cancer, and others. In one preferred embodiment of the invention, the cancer-associated sequences described herein have been found particularly advantageous in the detection of lung cancer due to their demonstrated complementation and high tumor coverage.

Essentially any biological sample suspected of containing cancer-associated markers, antibodies to such cancer-associated markers and/or cancer cells expressing such markers or antibodies may be used for the methods of the invention. For example, the biological sample can be a tissue sample, such as a tissue biopsy sample, known or suspected of containing cancer cells. The biological sample may be derived from a tissue suspected of being the site of origin of a primary tumor. Alternatively, the biological sample may be derived from a tissue or other biological sample distinct from the suspected site of origin of a primary tumor in order to detect the presence of metastatic cancer cells in the tissue or sample that have escaped the site of origin of the primary tumor. In certain embodiments, the biological sample is a tissue biopsy sample derived from tissue of the lung, pancreas, kidney, bladder or breast. In other embodiments, the biological sample tested according to such methods is selected from the group consisting of a biopsy sample, lavage sample, sputum sample, serum sample, peripheral blood sample, lymph node sample, bone marrow sample, urine sample, and pleural effusion sample.

A predetermined cut-off value used in the methods described herein for determining the presence of cancer can be readily identified using well-known techniques. For example, in one illustrative embodiment, the predetermined cut-off value for the detection of cancer is the average mean signal obtained when the relevant method of the invention is performed on suitable negative control samples, e.g., samples from patients without cancer. In another illustrative embodiment, a sample generating a signal that is at least two or three standard deviations above the predetermined cut-off value is considered positive.

In addition to definitions provided elsewhere in the specification, some terms have been defined as follows. Unless indicated or defined otherwise, all scientific and technical terms used herein have the same meaning as commonly understood by those skilled in the relevant art. General definitions of many terms used herein are provided in: Dictionary of Microbiology and Molecular Biology, 2nd ed. (Singleton et al., 1994, John Wiley & Sons, New York, N.Y.); The Harper Collins Dictionary of Biology (Hale & Marham, 1991, Harper Perennial, New York, N.Y.); and, Dorland's Illustrated Medical Dictionary, 27th ed. (W. A. Dorland, 1988, W. B. Saunders Co., Philadelphia, Pa.).

As discussed in further detail herein, the present invention achieves these and other related objectives by providing a methodology for the simultaneous detection of more than one polynucleotide, the presence of which is diagnostic of the presence of lung cancer cells in a biological sample. Certain of the various cancer detection methodologies disclosed herein have in common a step of hybridizing one or more oligonucleotide primers and/or probes, the hybridization of which is demonstrative of the presence of a tumor- and/or tissue-specific polynucleotide. Depending on the precise application contemplated, it may be preferred to employ one or more intron-spanning oligonucleotides that are inoperative against polynucleotide of genomic DNA and, thus, these oligonucleotides are effective in substantially reducing and/or eliminating the detection of genomic DNA in the biological sample.

Further disclosed herein are methods for enhancing the sensitivity of these detection methodologies by subjecting the biological samples to be tested to one or more cell capture and/or cell depletion methodologies.

By certain embodiments of the present invention, the presence of lung cancer cell in a patient may be determined by employing the following steps: (a) contacting a biological sample obtained from the patient with two or more oligonucleotides that hybridize to two or more polynucleotides that encode two or more lung tumor proteins as described herein; (b) detecting in the sample a level of at least one of the polynucleotides (such as, for example, mRNA) that hybridize to the oligonucleotides; and (c) comparing the level of polynucleotides that hybridize to the oligonucleotides with a predetermined cut-off value, and therefrom determining the presence or absence of lung cancer in the patient.

To permit hybridization under assay conditions, oligonucleotide primers and probes should comprise an oligonucleotide sequence that has at least about 60%, preferably at least about 75% and more preferably at least about 90%, identity to a portion of a polynucleotide encoding a lung tumor protein that is at least 10 nucleotides, and preferably at least 20 nucleotides, in length. Preferably, oligonucleotide primers hybridize to a polynucleotide encoding a polypeptide described herein under moderately stringent conditions, as defined above. Oligonucleotide primers which may be usefully employed in the diagnostic methods described herein preferably are at least 1040 nucleotides in length. In a preferred embodiment, the oligonucleotide primers comprise at least 10 contiguous nucleotides, more preferably at least 15 contiguous nucleotides, of a DNA molecule having a sequence recited in SEQ ID NO: 1, 3, 5 or 7. Techniques for both PCR based assays and hybridization assays are well known in the art (see, for example, Mullis et al., Cold Spring Harbor Symp. Quant. Biol., 51:263, 1987; Erlich ed., PCR Technology, Stockton Press, NY, 1989).

The present invention also provides amplification-based methods for detecting the presence of lung cancer cells in a patient. Exemplary methods comprise the steps of (a) obtaining a biological sample from the patient; (b) contacting the biological sample with two or more oligonucleotide pairs specific for independent polynucleotide sequences which are unrelated to one another, wherein the oligonucleotide pairs hybridize, under moderately stringent conditions, to their respective polynucleotides and the complements thereof (c) amplifying the polynucleotides; and (d) detecting the amplified polynucleotides; wherein the presence of one or more of the amplified polynucleotides indicates the presence of lung cancer cells in the patient.

Methods according to the present invention are suitable for identifying polynucleotides obtained from a wide variety of biological sample such as, for example, blood, serum, lymph node, bone marrow, sputum, urine and tumor biopsy sample, among others.

Certain exemplary embodiments of the present invention provide methods wherein the polynucleotides to be detected are selected from the group consisting of L762, L984, L550, L552, L763 and L587. Alternatively and/or additionally, polynucleotides to be detected may be selected from the group consisting of those depicted in SEQ ID NOs: 1, 3, 5, 7, 21 and 26.

Suitable exemplary oligonucleotide probes and/or primers that may be used according to the methods of the present invention are disclosed herein. In certain preferred embodiments that eliminate the background detection of genomic DNA, the oligonucleotides may be intron spanning oligonucleotides.

Depending on the precise application contemplated, the artisan may prefer to detect the tissue- and/or tumor-specific polynucleotides by detecting a radiolabel and detecting a fluorophore. More specifically, the oligonucleotide probe and/or primer may comprises a detectable moiety such as, for example, a radiolabel and/or a fluorophore.

Alternatively or additionally, methods of the present invention may also comprise a step of fractionation prior to detection of the tissue- and/or tumor-specific polynucleotides such as, for example, by gel electrophoresis.

In other embodiments, methods described herein may be used as to monitor the progression of cancer. By these embodiments, assays as provided for the diagnosis of lung cancer may be performed over time, and the change in the level of reactive polypeptide(s) or polynucleotide(s) evaluated. For example, the assays may be performed every 24-72 hours for a period of 6 months to 1 year, and thereafter performed as needed. In general, a cancer is progressing in those patients in whom the level of polypeptide or polynucleotide detected increases over time. In contrast, the cancer is not progressing when the level of reactive polypeptide or polynucleotide either remains constant or decreases with time.

Certain in vivo diagnostic assays may be performed directly on a tumor. One such assay involves contacting tumor cells with a binding agent. The bound binding agent may then be detected directly or indirectly via a reporter group. Such binding agents may also be used in histological applications. Alternatively, polynucleotide probes may be used within such applications.

As noted above, to improve sensitivity, multiple lung tumor protein markers may be assayed within a given sample. It will be apparent that binding agents specific for different proteins provided herein may be combined within a single assay. Further, multiple primers or probes may be used concurrently. The selection of tumor protein markers may be based on routine experiments to determine combinations that results in optimal sensitivity. In addition, or alternatively, assays for tumor proteins provided herein may be combined with assays for other known tumor antigens.

The above descriptions are intended to be exemplary only. It will be recognized that numerous other assays exist that can be used for amplifying and/or detecting mRNA expression in biological samples. Such methods are also considered within the scope of the present invention.

According to another aspect, the present invention provides methods, compositions and kits employing binding agents, such as antibodies or antigen-binding fragments thereof, that specifically bind the cancer-associated L762P, L550S, L587S and/or L984P polypeptide sequences disclosed herein or a portion, variant or derivative thereof. Such binding agents may be used in the methods of the invention for detecting the presence and/or level of L762P, L550S, L587S and/or L984P polypeptide expression in biological samples (including tissue sections) using representative assays either illustratively described herein or known and available in the art.

A binding agent used according to this aspect of the invention can include essentially any binding agent having sufficient specificity and affinity for the cancer-associated markers described herein to facilitate the detection and identification of the markers in a biological sample. For example, by way of illustration, a binding agent may be an antibody, an antigen-binding fragment of an antibody, a ribosome, with or without a peptide component, an RNA molecule, or a polypeptide. In one illustrative example, a binding agent is an agent identified via phage display library screening to specifically bind a cancer-associated marker described herein.

Certain preferred binding agents for use according to the present invention include antibodies or antigen-binding fragments thereof that specifically bind a cancer-associated marker described herein. An antibody or antigen-binding fragment thereof is said to “specifically bind” to a polypeptide of the invention if it reacts at a detectable level (within, for example, an ELISA) with the polypeptide but does not react with a biologically unrelated polypeptide in any statistically significant fashion under the same or similar conditions. Specific binding, as used in this context, generally refers to the non-covalent interactions of the type that occur between an immunoglobulin molecule and an antigen for which the immunoglobulin is specific. The strength or affinity of immunological binding interactions can be expressed in terms of the dissociation constant (K_{d}) of the interaction, wherein a smaller K_{d} represents a greater affinity. Immunological binding properties of selected polypeptides can be quantified using methods well-known in the art. One such method entails measuring the rates of antigen-binding site/antigen complex formation and dissociation, wherein those rates depend on the concentrations of the complex partners, the affinity of the interaction, and the geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity and is thus equal to the dissociation constant K_{d}. See, generally, Davies et al. (1990) Annual Rev. Biochem. 59:439-473.

An “antigen-binding site” or “binding portion” of an antibody refers to the part of the immunoglobulin molecule that participates in antigen binding. The antigen-binding site is formed by amino acid residues of the N-terminal variable (V) regions of the heavy (H) and light (L) chains. Three highly divergent stretches within the variable regions of the heavy and light chains are referred to as “hypervariable regions.” These hypervariable regions are interposed between more conserved flanking stretches known as “framework regions” (FRs). Thus, the term “FR” refers to amino acid sequences naturally found between and adjacent to hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen. The three hypervariable regions of each of the heavy and light chains are referred to as “complementarity-determining regions” (CDRs).

In one embodiment, antibodies or other binding agents that bind to a cancer-associated marker described herein will preferably generate a signal indicating the presence of a cancer in at least about 20%, 30% or 50% of samples and/or patients with the disease. Biological samples (e.g., blood, sera, sputum, urine and/or tumor biopsies) from patients with and without a cancer (as determined using standard clinical tests) may be assayed as described herein for the presence of polypeptides that bind to the binding agent.

In one preferred embodiment, a binding agent is an antibody or an antigen-binding fragment thereof. Antibodies may be prepared by any of a variety of techniques known to those of ordinary skill in the art (see, e.g., Harlow and Lane, Antibodies: A Laboratory Manual, Cold Spring Harbor Laboratory, 1988). Illustrative methods for the production of antibodies generally involve the use of a polypeptide, produced by either recombinant or synthetic approaches, as an immunogen. In order to produce a desired recombinant polypeptide, a nucleotide sequence encoding the polypeptide, or functional equivalents, may be inserted into an appropriate expression vector, i.e., a vector which contains the necessary elements for the transcription and translation of the inserted coding sequence. Methods which are well-known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described, for example, in: Sambrook, J. et al. (1989) Molecular Cloning, A Laboratory Manual, Cold Spring Harbor Press, Plainview, N.Y.; and, Ausubel, F. M. et al. (1989) Current Protocols in Molecular Biology, John Wiley & Sons, New York, N.Y.

A variety of expression vector/host systems may be utilized to contain and express polynucleotide sequences. These include, but are not limited to: microorganisms, such as bacteria, transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or bacterial expression vectors (e.g., Ti or pBR322 plasmids); and, animal cell systems. These and other suitable expression systems for the production of recombinant polypeptides are known in the art and may be used in the practice of the present invention.

In addition to recombinant production methods, peptide and/or polypeptides may be synthesized, in whole or in part, using chemical methods well-known in the art (see Caruthers, M. H. et al. (1980) Nucl. Acids Res. Symp. Ser. 215-223, Horn, T. et al. (1980) Nucl. Acids Res. Symp. Ser. 225-232). For example, peptide synthesis can be performed using various solid-phase techniques (Roberge, J. Y. et al. (1995) Science 269:202-204) and automated synthesis may be achieved, for example, using the ABI 431A Peptide Synthesizer (Perkin Elmer, Palo Alto, Calif.). A newly synthesized peptide may be substantially purified by preparative HPLC (e.g., Creighton, T. (1983) Proteins, Structures and Molecular Principles, WH Freeman and Co., New York, N.Y.) or other comparable techniques available in the art. The composition of the synthetic peptides may be confirmed by amino acid analysis or sequencing (e.g., the Edman degradation procedure). Additionally, the amino acid sequence of a polypeptide, or any part thereof, may be altered during direct synthesis and/or combined using chemical methods with sequences from other proteins, or any part thereof, to produce a variant polypeptide.

In certain embodiments, antibodies can be produced by cell culture techniques, including the generation of monoclonal antibodies as described herein, or via transfection of antibody genes into suitable bacterial or mammalian cell hosts in order to allow for the production of recombinant antibodies. In one technique, an immunogen comprising a polypeptide is initially injected into any of a wide variety of mammals (e.g., mice, rats, rabbits, sheep or goats). In this step, the polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for a polypeptide of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized, for example, by fusion with a myeloma cell fusion partner, preferably one that is syngeneic with the immunized animal. A variety of fusion techniques may be employed. For example, the spleen cells and myeloma cells may be combined with a non-ionic detergent for a few minutes and then plated at low density on a selective medium that supports the growth of hybrid cells but not myeloma cells. One illustrative selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

A number of “humanized” antibody molecules comprising an antigen-binding site derived from a non-human immunoglobulin have been described, including chimeric antibodies having rodent V regions and their associated CDRs fused to human constant domains (Winter et al. (1991) Nature 349:293-299; Lobuglio et al. (1989) Proc. Nat. Acad. Sci. USA 86:4220-4224; Shaw et al. (1987) J Immunol. 138:4534-4538; and, Brown et al. (1987) Cancer Res. 47:3577-3583), rodent CDRs grafted into a human supporting FR prior to fusion with an appropriate human antibody constant domain (Riechmann et al. (1988) Nature 332:323-327; Verhoeyen et al. (1988) Science 239:1534-1536; and, Jones et al. (1986) Nature 321:522-525), and rodent CDRs supported by recombinantly veneered rodent FRs (European Patent No. 0 519 596). These “humanized” molecules are designed to minimize unwanted immunological response toward rodent anti-human antibody molecules.

A variety of protocols for detecting and/or measuring the level of expression of polypeptides, using either polyclonal or monoclonal antibodies specific for the product, are known in the art. Examples include enzyme-linked immunosorbent assay (ELISA), immunohistochemistry (IHC), radioimmunoassay (RIA), fluorescence activated cell sorting (FACS), and the like. A two-site, monoclonal-based immunoassay utilizing monoclonal antibodies reactive to two non-interfering epitopes on a given polypeptide may be preferred for some applications, but a competitive binding assay may also be employed. These and other assays are described, among other places, in Hampton, R. et al. (1990; Serological Methods, a Laboratory Manual, APS Press, St Paul. Minn.); Maddox, D. E. et al. (1983; J. Exp. Med. 158:1211-1216); and, Harlow and Lane (Antibodies: A Laboratory Manual, Cold Spring Harbor Laboratory, 1988).

In general, the presence or absence of a cancer in a patient may be determined by (a) contacting a biological sample obtained from a patient with binding agents specific for at least two of the cancer-associated markers selected from the group consisting of L762P, L550S, L587S and L984P; (b) detecting in the sample a level of polypeptide that binds to each binding agent; and, (c) comparing the level of polypeptide with a predetermined cut-off value, wherein a level of polypeptide present in a biological sample that is above the predetermined cut-off value for one or more marker is indicative of the presence of cancer cells in the biological sample.

In one illustrative embodiment, the assay involves the use of binding agent immobilized on a solid support to bind to and remove the polypeptide from the remainder of the sample. The bound polypeptide may then be detected using a detection reagent that contains a reporter group and specifically binds to the binding agent/polypeptide complex. Such detection reagents may comprise, for example, a binding agent that specifically binds to the polypeptide or an antibody or other agent that specifically binds to the binding agent, such as an anti-immunoglobulin, protein G, protein A or a lectin. Alternatively, a competitive assay may be utilized in which a polypeptide is labeled with a reporter group and allowed to bind to the immobilized binding agent after incubation of the binding agent with the sample. The extent to which components of the sample inhibit the binding of the labeled polypeptide to the binding agent is indicative of the reactivity of the sample with the immobilized binding agent. Suitable polypeptides for use within such assays include full length proteins and polypeptide portions thereof to which the binding agent binds, as described above.

The solid support may be any material known to those of ordinary skill in the art to which the protein may be attached. For example, the solid support may be a test well in a microtiter plate or a nitrocellulose or other suitable membrane. Alternatively, the support may be a bead or disc, such as glass, fiberglass, latex, or a plastic material such as polystyrene or polyvinylchloride. The support may also be a magnetic particle or a fiber optic sensor, such as those disclosed, for example, in U.S. Pat. No. 5,359,681. The binding agent may be immobilized on the solid support using a variety of techniques known to those of skill in the art, which are amply described in the patent and scientific literature. In the context of the present invention, the term “immobilization” refers to both noncovalent association, such as adsorption, and covalent attachment which may be a direct linkage between the agent and functional groups on the support or may be a linkage by way of a cross-linking agent. Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of binding agent ranging from about 10 ng to about 10 μg, and preferably about 100 ng to about 1 μg, is sufficient to immobilize an adequate amount of binding agent.

Covalent attachment of binding agent to a solid support may generally be achieved by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12-A13).

In certain embodiments, the assay is a two-antibody sandwich assay. This assay may be performed by first contacting an antibody that has been immobilized on a solid support, commonly the well of a microtiter plate, with the sample, such that polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

More specifically, once the antibody is immobilized on the support as described above, the remaining protein binding sites on the support are typically blocked. Any suitable blocking agent known to those of ordinary skill in the art, such as bovine serum albumin or Tween 20™ (Sigma Chemical Co., St. Louis, Mo.). The immobilized antibody is then incubated with the sample and polypeptide is allowed to bind to the antibody. The sample may be diluted with a suitable diluent, such as phosphate-buffered saline (PBS), prior to incubation. In general, an appropriate contact time (i.e., incubation time) is a period of time that is sufficient to detect the presence of polypeptide within a sample obtained from an individual with cancer. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. The second antibody, which contains a reporter group, may then be added to the solid support. Preferred reporter groups include those groups recited above as well as other known in the art.

The detection reagent is then incubated with the immobilized antibody-polypeptide complex for an amount of time sufficient to detect the bound polypeptide. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of a cancer, such as lung cancer, the signal detected from the reporter group that remains bound to the solid support is generally compared to a signal that corresponds to a predetermined cut-off value. In one embodiment, the cut-off value for the detection of a cancer is the average mean signal obtained when the immobilized antibody is incubated with samples from patients without the cancer. In another embodiment, a sample generating a signal that is three standard deviations above the predetermined cut-off value is considered positive for the cancer. In another embodiment, the cut-off value is determined using a Receiver Operator Curve, according to the method of Sackett et al., Clinical Epidemiology: A Basic Science for Clinical Medicine, Little Brown and Co., 1985, p. 106-7. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a cancer.

In a related embodiment, the assay is performed in a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described above. In the strip test format, one end of the membrane to which binding agent is bound is immersed in a solution containing the sample. The sample migrates along the membrane through a region containing second binding agent and to the area of immobilized binding agent. Concentration of second binding agent at the area of immobilized antibody indicates the presence of a cancer. Typically, the concentration of second binding agent at that site generates a pattern, such as a line, that can be read visually. The absence of such a pattern indicates a negative result. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in such assays are antibodies and antigen-binding fragments thereof. In certain embodiments, the amount of antibody immobilized on the membrane ranges from about 25 ng to about 1 μg, and in other embodiments is from about 50 ng to about 500 ng. Such tests can typically be performed with a very small amount of biological sample.

In other embodiments of the invention, the cancer-associated polypeptides described herein may be utilized to detect the presence of antibodies specific for the polypeptides in a biological sample. The detection of such antibodies specific for cancer-associated polypeptides may be indicative of the presence of cancer in the patient from which the biological sample was derived. In one illustrative example, a biological sample is contacted with a solid phase to which at least two cancer-associated polypeptides, such as recombinant or synthetic L762P, L550S, L587S and/or L984P polypeptides, or portions thereof, have been attached. In certain other embodiments, the cancer-associated polypeptides used in this aspect of the invention comprise at least two polypeptides, or portions thereof, selected from the group consisting of an L762P protein having an amino acid sequence set forth in SEQ ID NO: 2, the L550S protein comprises an amino acid sequence set forth in SEQ ID NO:23, an L587S protein having an amino acid sequence set forth in SEQ ID NO: 5, and/or an L984P protein having an amino acid sequence set forth in SEQ ID NO: 7. In one illustrative embodiment, the biological sample tested according to this aspect of the invention is a peripheral blood sample. A biological sample is generally contacted with the polypeptides for a time and under conditions sufficient to form detectable antigen/antibody complexes. Indicator reagents may be used to facilitate detection, depending upon the assay system chosen. In another embodiment, a biological sample is contacted with a solid phase to which a recombinant or synthetic polypeptide is attached and is also contacted with a monoclonal or polyclonal antibody specific for the polypeptide, which preferably has been labeled with an indicator reagent. After incubation for a time and under conditions sufficient for antibody/antigen complexes to form, the solid phase is separated from the free phase and the label is detected in either the solid or free phase as an indication of the presence of antibodies. Other assay formats utilizing recombinant and/or synthetic polypeptides for the detection of antibodies are available in the art and may be employed in the practice of the present invention.

Cell Enrichment

In other aspects of the present invention, cell capture technologies may be used prior to polynucleotide detection to improve the sensitivity of the various detection methodologies disclosed herein.

Exemplary cell enrichment methodologies employ immunomagnetic beads that are coated with specific monoclonal antibodies to surface cell markers, or tetrameric antibody complexes, may be used to first enrich or positively select cancer cells in a sample. Various commercially available kits may be used, including Dynabeads® Epithelial Enrich (Dynal Biotech, Oslo, Norway), StemSep™ (StemCell Technologies, Inc., Vancouver, BC), and RosetteSep (StemCell Technologies). The skilled artisan will recognize that other readily available methodologies and kits may also be suitably employed to enrich or positively select desired cell populations.

Dynabeads® Epithelial Enrich contains magnetic beads coated with mAbs specific for two glycoprotein membrane antigens expressed on normal and neoplastic epithelial tissues. The coated beads may be added to a sample and the sample then applied to a magnet, thereby capturing the cells bound to the beads. The unwanted cells are washed away and the magnetically isolated cells eluted from the beads and used in further analyses.

RosetteSep can be used to enrich cells directly from a blood sample and consists of a cocktail of tetrameric antibodies that target a variety of unwanted cells and crosslinks them to glycophorin A on red blood cells (RBC) present in the sample, forming rosettes. When centrifuged over Ficoll, targeted cells pellet along with the free RBC.

The combination of antibodies in the depletion cocktail determines which cells will be removed and consequently which cells will be recovered. Antibodies that are available include, but are not limited to: CD2, CD3, CD4, CD5, CD8, CD10, CD11b, CD14, CD15, CD16, CD19, CD20, CD24, CD25, CD29, CD33, CD34, CD36, CD38, CD41, CD45, CD45RA, CD45RO, CD56, CD66B, CD66e, HLA-DR, IgE, and TCRαβ. Additionally, it is contemplated in the present invention that mAbs specific for lung tumor antigens, can be developed and used in a similar manner. For example, mAbs that bind to tumor-specific cell surface antigens may be conjugated to magnetic beads, or formulated in a tetrameric antibody complex, and used to enrich or positively select metastatic lung tumor cells from a sample. Such a system can be used to evaluate blood samples from different forms of lung cancers, in particular adneo and squamous forms of NSCLC and small cell carcinomas for the presence of circulating tumor cells using the inventive multiplex PCR assay as described herein.

Once a sample is enriched or positively selected, cells may be further analyzed. For example, the cells may be lysed and RNA isolated. RNA may then be subjected to RT-PCR analysis using lung tumor-specific multiplex primers in a Real-time PCR assay as described herein.

In another aspect of the present invention, cell capture technologies may be used in conjunction with Real-Time PCR to provide a more sensitive tool for detection of metastatic cells expressing lung tumor antigens.

Yet another method that may be employed is an anti-ganglioside G_{M1}/G_{M1} cell capture antibody system. Gangliosides are cell membrane bound glycosphingolipids, several species of which have been shown to be over-expressed on the cell surface of most cancers of neuroectodermal and epithelial origin, in particular lung cancer. Cell surface expression of G_{M2} is seen in several types of lung cancer, particularly in SCLC which make it an attractive target for a monoclonal antibody based lung cancer immunotherapy and also for use as a capture method in conjunction with G_{M1}.

Probes and Primers

As noted above and as described in further detail herein, certain methods, compositions and kits according to the present invention utilize two or more oligonucleotide primer pairs for the detection of lung cancer. The ability of such nucleic acid probes to specifically hybridize to a sequence of interest will enable them to be of use in detecting the presence of complementary sequences in a biological sample.

By “oligonucleotide” is meant a polymeric chain of two or more chemical subunits, each subunit comprising a nucleotide base moiety, a sugar moiety, and a linking moiety that joins the subunits in a linear spacial configuration. An oligonucleotide may contain up to thousands of such subunits, but generally contains subunits in a range having a lower limit of between about 5 to about 10 subunits, and an upper limit of between about 20 to about 1,000 subunits. The most common nucleotide base moieties are guanine (G), adenine (A), cytosine (C), thymine (T) and uracil (U), although other rare or modified nucleotide bases able to form hydrogen bonds (e.g., inosine (I)) are well-known to those skilled in the art. The most common sugar moieties are ribose and deoxyribose, although 2′-O-methyl ribose, halogenated sugars, and other modified and different sugars are well-known. The linking group is usually a phosphorus-containing moiety, commonly a phosphodiester linkage, although other known phosphate-containing linkages (e.g., phosphorothioates or methylphosphonates) and non-phosphorus-containing linkages (e.g., peptide-like linkages found in “peptide nucleic acids” or PNAs) known in the art are included. Likewise, an oligonucleotide includes one in which at least one base moiety has been modified, for example, by the addition of propyne groups, so long as: (1) the modified base moiety retains the ability to form a non-covalent association with G, A, C, T or U; and, (2) an oligonucleotide comprising at least one modified nucleotide base moiety is not sterically prevented from hybridizing with a complementary single-stranded nucleic acid. An oligonucleotide's ability to hybridize with a complementary nucleic acid strand under particular conditions (e.g., temperature or salt concentration) is governed by the sequence of base moieties, as is well-known to those skilled in the art (Sambrook, J. et al., 1989, Molecular Cloning, A Laboratory Manual, 2nd ed. (Cold Spring Harbor Laboratory Press, Cold Spring Harbor, N.Y.), particularly pp. 7.37-7.57 and 11.47-11.57).

By “primer” or “amplification primer” is meant an oligonucleotide capable of binding to a region of a target nucleic acid or its complement and promoting, either directly or indirectly, nucleic acid amplification of the target nucleic acid. In most cases, a primer will have a free 3′ end that can be extended by a nucleic acid polymerase. All amplification primers include a base sequence capable of hybridizing via complementary base interactions to at least one strand of the target nucleic acid or a strand that is complementary to the target sequence. For example, in PCR, amplification primers anneal to opposite strands of a double-stranded target DNA that has been denatured. The primers are extended by a thermostable DNA polymerase to produce double-stranded DNA products, which are then denatured with heat, cooled and annealed to amplification primers. Multiple cycles of the foregoing steps (e.g., about 20 to about 50 thermic cycles) exponentially amplifies the double-stranded target DNA.

The term “specific for” in the context of primers and probes, is a term of art well understood by the skilled artisan to refer to a particular primer or probe capable of annealing/hybridizing/binding to a target nucleic acid or its complement but which primer or probe does not detectably anneal/hybridize/bind to non-target nucleic acid sequences under the same conditions. Thus, for example, in the setting of an amplification technique, a primer, primer set, or probe that is specific for a target nucleic acid of interest would amplify the target nucleic acid of interest but would not detectably amplify sequences that are not of interest. As would be recognized by the skilled artisan, primers and probes that are specific for a particular target nucleic acid sequence of interest can be designed using any of a variety of computer programs available in the art (see, e.g., Methods Mol Biol. 2002; 192:19-29) or can be designed by eye by comparing the nucleic acid sequence of interest to other relevant known sequences. In certain embodiments, the conditions under which a primer or probe is specific for a target nucleic acid of interest can be routinely optimized by changing parameters of the reaction conditions. For example, in PCR, a variety of parameters can be changed, such as annealing or extension temperature, concentration of primer and/or probe, magnesium concentration, the use of “hot start” conditions such as wax beads or specifically modified polymerase enzymes, addition of formamide, DMSO or other similar compounds. In other hybridization methods, conditions can similarly be routinely optimized by the skilled artisan using techniques known in the art.

Alternatively, in other embodiments, the probes and/or primers of the present invention may be employed for detection via nucleic acid hybridization. As such, it is contemplated that nucleic acid segments that comprise a sequence region of at least about 15 nucleotide long contiguous sequence that has the same sequence as, or is complementary to, a 15 nucleotide long contiguous sequence of a polynucleotide to be detected will find particular utility. Longer contiguous identical or complementary sequences, e.g., those of about 20, 30, 40, 50, 100, 200, 500, 1000 (including all intermediate lengths) and even up to full length sequences will also be of use in certain embodiments.

Oligonucleotide primers having sequence regions consisting of contiguous nucleotide stretches of 10-14, 15-20, 30, 50, or even of 100-200 nucleotides or so (including intermediate lengths as well), identical or complementary to a polynucleotide to be detected, are particularly contemplated as primers for use in amplification reactions such as, e.g., the polymerase chain reaction (PCR™). This would allow a polynucleotide to be analyzed, both in diverse biological samples such as, for example, blood, lymph nodes and bone marrow.

The use of a primer of about 15-25 nucleotides in length allows the formation of a duplex molecule that is both stable and selective. Molecules having contiguous complementary sequences over stretches greater than 15 bases in length are generally preferred, though, in order to increase stability and selectivity of the hybrid, and thereby improve the quality and degree of specific hybrid molecules obtained. One will generally prefer to design primers having gene-complementary stretches of 15 to 25 contiguous nucleotides, or even longer where desired.

Primers may be selected from any portion of the polynucleotide to be detected. All that is required is to review the sequence, such as those exemplary polynucleotides set forth herein or to any continuous portion of the sequence, from about 15-25 nucleotides in length up to and including the full length sequence, that one wishes to utilize as a primer. The choice of primer sequences may be governed by various factors. For example, one may wish to employ primers from towards the termini of the total sequence. The exemplary primers disclosed herein may optionally be used for their ability to selectively form duplex molecules with complementary stretches of the entire polynucleotide of interest such as those set forth SEQ ID NOs: 1, 3, 5, 7, 21 and 26.

The present invention further provides the nucleotide sequence of various exemplary oligonucleotide primers and probes, that may be used, as described in further detail herein, according to the methods of the present invention for the detection of cancer.

Oligonucleotide primers according to the present invention may be readily prepared routinely by methods commonly available to the skilled artisan including, for example, directly synthesizing the primers by chemical means, as is commonly practiced using an automated oligonucleotide synthesizer. Depending on the application envisioned, one will typically desire to employ varying conditions of hybridization to achieve varying degrees of selectivity of probe towards target sequence. For applications requiring high selectivity, one will typically desire to employ relatively stringent conditions to form the hybrids, e.g., one will select relatively low salt and/or high temperature conditions, such as provided by a salt concentration of from about 0.02 M to about 0.15 M salt at temperatures of from about 50° C. to about 70° C. Such selective conditions tolerate little, if any, mismatch between the probe and the template or target strand, and would be particularly suitable for isolating related sequences.

Polynucleotide Amplification Techniques

Each of the specific embodiments outlined herein for the detection of lung cancer has in common the detection of a tissue- and/or tumor-specific polynucleotide via the hybridization of one or more oligonucleotide primers and/or probes. Depending on such factors as the relative number of cancer cells present in the biological sample and/or the level of polynucleotide expression within each lung cancer cell, it may be preferred to perform an amplification step prior to performing the steps of detection. For example, at least two oligonucleotide primers may be employed in a polymerase chain reaction (PCR) based assay to amplify a portion of a lung tumor cDNA derived from a biological sample, wherein at least one of the oligonucleotide primers is specific for (i.e., hybridizes to) a polynucleotide encoding the lung tumor protein. The amplified cDNA may optionally be subjected to a fractionation step such as, for example, gel electrophoresis.

By “amplification” or “nucleic acid amplification” is meant production of multiple copies of a target nucleic acid that contains at least a portion of the intended specific target nucleic acid sequence (e.g., L762P, L550S, L587S and/or L984P). The multiple copies may be referred to as amplicons or amplification products. In certain embodiments, the amplified target contains less than the complete target gene sequence (introns and exons) or an expressed target gene sequence (spliced transcript of exons and flanking untranslated sequences). For example, specific amplicons may be produced by amplifying a portion of the target polynucleotide by using amplification primers that hybridize to, and initiate polymerization from, internal positions of the target polynucleotide. Preferably, the amplified portion contains a detectable target sequence that may be detected using any of a variety of well-known methods.

Many well-known methods of nucleic acid amplification require thermocycling to alternately denature double-stranded nucleic acids and hybridize primers; however, other well-known methods of nucleic acid amplification are isothermal. The polymerase chain reaction (U.S. Pat. Nos. 4,683,195; 4,683,202; 4,800,159; 4,965,188), commonly referred to as PCR, uses multiple cycles of denaturation, annealing of primer pairs to opposite strands, and primer extension to exponentially increase copy numbers of the target sequence. In a variation called RT-PCR, reverse transcriptase (RT) is used to make a complementary DNA (cDNA) from mRNA, and the cDNA is then amplified by PCR to produce multiple copies of DNA. The ligase chain reaction (Weiss, R. 1991, Science 254: 1292), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product. Another method is strand displacement amplification (Walker, G. et al., 1992, Proc. Natl. Acad. Sci. USA 89:392-396; U.S. Pat. Nos. 5,270,184 and 5,455,166), commonly referred to as SDA, which uses cycles of annealing pairs of primer sequences to opposite strands of a target sequence, primer extension in the presence of a dNTPαS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (European Pat. No. 0 684 315). Other amplification methods include: nucleic acid sequence based amplification (U.S. Pat. No. 5,130,238), commonly referred to as NASBA; one that uses an RNA replicase to amplify the probe molecule itself (Lizardi, P. et al., 1988, BioTechnol. 6: 1197-1202), commonly referred to as Qβ replicase; a transcription based amplification method (Kwoh, D. et al., 1989, Proc. Natl. Acad. Sci. USA 86:1173-1177); self-sustained sequence replication (Guatelli, J. et al., 1990, Proc. Natl. Acad. Sci. USA 87: 1874-1878); and, transcription mediated amplification (U.S. Pat. Nos. 5,480,784 and 5,399,491), commonly referred to as TMA. For further discussion of known amplification methods see Persing, David H., 1993, “In Vitro Nucleic Acid Amplification Techniques” in Diagnostic Medical Microbiology: Principles and Applications (Persing et al., Eds.), pp. 51-87 (American Society for Microbiology, Washington, D.C.).

Illustrative transcription-based amplification systems of the present invention include TMA, which employs an RNA polymerase to produce multiple RNA transcripts of a target region (U.S. Pat. Nos. 5,480,784 and 5,399,491). TMA uses a “promoter-primer” that hybridizes to a target nucleic acid in the presence of a reverse transcriptase and an RNA polymerase to form a double-stranded promoter from which the RNA polymerase produces RNA transcripts. These transcripts can become templates for further rounds of TMA in the presence of a second primer capable of hybridizing to the RNA transcripts. Unlike PCR, LCR or other methods that require heat denaturation, TMA is an isothermal method that uses an RNase H activity to digest the RNA strand of an RNA:DNA hybrid, thereby making the DNA strand available for hybridization with a primer or promoter-primer. Generally, the RNase H activity associated with the reverse transcriptase provided for amplification is used.

In an illustrative TMA method, one amplification primer is an oligonucleotide promoter-primer that comprises a promoter sequence which becomes functional when double-stranded, located 5′ of a target-binding sequence, which is capable of hybridizing to a binding site of a target RNA at a location 3′ to the sequence to be amplified. A promoter-primer may be referred to as a “T7-primer” when it is specific for T7 RNA polymerase recognition. Under certain circumstances, the 3′ end of a promoter-primer, or a subpopulation of such promoter-primers, may be modified to block or reduce primer extension. From an unmodified promoter-primer, reverse transcriptase creates a cDNA copy of the target RNA, while RNase H activity degrades the target RNA. A second amplification primer then binds to the cDNA. This primer may be referred to as a “non-T7 primer” to distinguish it from a “T7-primer”. From this second amplification primer, reverse transcriptase creates another DNA strand, resulting in a double-stranded DNA with a functional promoter at one end. When double-stranded, the promoter sequence is capable of binding an RNA polymerase to begin transcription of the target sequence to which the promoter-primer is hybridized. An RNA polymerase uses this promoter sequence to produce multiple RNA transcripts (i.e., amplicons), generally about 100 to 1,000 copies. Each newly-synthesized amplicon can anneal with the second amplification primer. Reverse transcriptase can then create a DNA copy, while the RNase H activity degrades the RNA of this RNA:DNA duplex. The promoter-primer can then bind to the newly synthesized DNA, allowing the reverse transcriptase to create a double-stranded DNA, from which the RNA polymerase produces multiple amplicons. Thus, a billion-fold isothermic amplification can be achieved using two amplification primers.

By “nucleic acid amplification conditions” is meant environmental conditions, including salt concentration, temperature, the presence or absence of temperature cycling, the presence of a nucleic acid polymerase, nucleoside triphosphates, and cofactors, that are sufficient to permit the production of multiple copies of a target nucleic acid or its complementary strand using a nucleic acid amplification method.

A “target-binding sequence” of an amplification primer is the portion that determines target specificity because that portion is capable of annealing to the target nucleic acid strand or its complementary strand but does not detectably anneal to non-target nucleic acid strands under the same conditions. The complementary target sequence to which the target-binding sequence hybridizes is referred to as a primer-binding sequence. For primers or amplification methods that do not require additional functional sequences in the primer (e.g., PCR amplification), the primer sequence consists essentially of a target-binding sequence, whereas other methods (e.g., TMA or SDA) include additional specialized sequences adjacent to the target-binding sequence (e.g., an RNA polymerase promoter sequence adjacent to a target-binding sequence in a promoter-primer or a restriction endonuclease recognition sequence for an SDA primer). It will be appreciated by those skilled in the art that all of the primer and probe sequences of the present invention may be synthesized using standard in vitro synthetic methods. Also, it will be appreciated that those skilled in the art could modify primer sequences disclosed herein using routine methods to add additional specialized sequences (e.g., promoter or restriction endonuclease recognition sequences) to make primers suitable for use in a variety of amplification methods. Similarly, promoter-primer sequences described herein can be modified by removing the promoter sequences to produce amplification primers that are essentially target-binding sequences suitable for amplification procedures that do not use these additional functional sequences.

By “target sequence” is meant the nucleotide base sequence of a nucleic acid strand, at least a portion of which is capable of being detected using primers and/or probes in the methods as described herein, such as a labeled oligonucleotide probe. Primers and probes bind to a portion of a target sequence, which includes either complementary strand when the target sequence is a double-stranded nucleic acid.

By “equivalent RNA” is meant a ribonucleic acid (RNA) having the same nucleotide base sequence as a deoxyribonucleic acid (DNA) with the appropriate U for T substitution(s). Similarly, an “equivalent DNA” is a DNA having the same nucleotide base sequence as an RNA with the appropriate T for U substitution(s). It will be appreciated by those skilled in the art that the terms “nucleic acid” and “oligonucleotide” refer to molecular structures having either a DNA or RNA base sequence or a synthetic combination of DNA and RNA base sequences, including analogs thereof, which include “abasic” residues.

By “detecting” an amplification product is meant any of a variety of methods for determining the presence of an amplified nucleic acid, such as, for example, hybridizing a labeled probe to a portion of the amplified product. A labeled probe is an oligonucleotide that specifically binds to another sequence and contains a detectable group that may be, for example, a fluorescent moiety, chemiluminescent moiety, radioisotope, biotin, avidin, enzyme, enzyme substrate, or other reactive group. Preferably, a labeled probe includes an acridinium ester (AE) moiety that can be detected chemiluminescently under appropriate conditions (as described, e.g., in U.S. Pat. No. 5,283,174). Other well-known detection techniques include, for example, gel filtration, gel electrophoresis and visualization of the amplicons, and High Performance Liquid Chromatography (HPLC). The detecting step may either be qualitative or quantitative, although quantitative detection of amplicons may be preferred, as the level of gene expression may be indicative of the degree of metastasis, recurrence of cancer and/or responsiveness to therapy.

Assays for purifying and detecting a target polynucleotide often involve capturing a target polynucleotide on a solid support. The solid support retains the target polynucleotide during one or more washing steps of a target polynucleotide purification procedure. One technique involves capture of the target polynucleotide by a polynucleotide fixed to a solid support and hybridization of a detection probe to the captured target polynucleotide (e.g., U.S. Pat. No. 4,486,539). Detection probes not hybridized to the target polynucleotide are readily washed away from the solid support. Thus, remaining label is associated with the target polynucleotide initially present in the sample. Another technique uses a mediator polynucleotide that hybridizes to both a target polynucleotide and a polynucleotide fixed to a solid support such that the mediator polynucleotide joins the target polynucleotide to the solid support to produce a bound target (e.g., U.S. Pat. No. 4,751,177). A labeled probe can be hybridized to the bound target and unbound labeled probe can be washed away from the solid support.

The primers and probes of the present invention may be used in amplification and detection methods that use nucleic acid substrates isolated by any of a variety of well-known and established methodologies (e.g., Sambrook, J. et al., 1989, Molecular Cloning, A laboratory Manual, 2^{nd} ed. (Cold Spring Harbor Laboratory Press, Cold Spring Harbor, N.Y.), pp. 7.37-7.57; Lin, L. et al., 1993, “Simple and Rapid Sample Preparation Methods for Whole Blood and Blood Plasma” in Diagnostic Molecular Microbiology, Principles and Applications (Persing, D. H. et al., Eds., American Society for Microbiology, Washington, D.C.), pp. 605-616). In one illustrative example, the target mRNA may be prepared by the following procedure to yield mRNA suitable for use in amplification. Briefly, cells in a biological sample (e.g., peripheral blood or bone marrow cells) are lysed by contacting the cell suspension with a lysing solution containing at least about 150 mM of a soluble salt, such as lithium halide, a chelating agent and a non-ionic detergent in an effective amount to lyse the cellular cytoplasmic membrane without causing substantial release of nuclear DNA or RNA. The cell suspension and lysing solution are mixed at a ratio of about 1:1 to 1:3. The detergent concentration in the lysing solution is between about 0.5-1.5% (v/v). Any of a variety of known non-ionic detergents are effective in the lysing solution (e.g., TRITON®-type, TWEEN®-type and NP-type); typically, the lysing solution contains an octylphenoxy polyethoxyethanol detergent, preferably 1% TRITON® X-102. This procedure may advantageously with biological samples that contain cell suspensions (e.g., blood and bone marrow), but it works equally well on other tissues if the cells are separated using standard mincing, screening and/or proteolysis methods to separate cells individually or into small clumps. After cell lysis, the released total RNA is stable and may be stored at room temperature for at least 2 hours without significant RNA degradation without additional RNase inhibitors. Total RNA may be used in amplification without further purification or mRNA may be isolated using standard methods generally dependent on affinity binding to the poly-A portion of mRNA.

In certain embodiments, mRNA isolation employs capture particles consisting essentially of poly-dT oligonucleotides attached to insoluble particles. The capture particles are added to the above-described lysis mixture, the poly-dT moieties annealed to the poly-A mRNA, and the particles separated physically from the mixture. Generally, superparamagnetic particles may be used and separated by applying a magnetic field to the outside of the container. Preferably, a suspension of about 300 μg of particles (in a standard phosphate buffered saline (PBS), pH 7.4, of 140 mM NaCl) having either dT₁₄ or dT₃₀ linked at a density of about 1 to 100 pmoles per mg (preferably 10-100 pmols/mg, more preferably 10-50 pmols/mg) are added to about 1 mL of lysis mixture. Any superparamagnetic particles may be used, although typically the particles are a magnetite core coated with latex or silica (e.g., commercially available from Serodyn or Dynal) to which poly-dT oligonucleotides are attached using standard procedures (Lund et al., Nuc. Acids Res., 1988, 16:10861-10880). The lysis mixture containing the particles is gently mixed and incubated at about 22-42° C. for about 30 minutes, when a magnetic field is applied to the outside of the tube to separate the particles with attached mRNA from the mixture and the supernatant is removed. The particles are washed one or more times, generally three, using standard resuspension methods and magnetic separation as described above. Then, the particles are suspended in a buffer solution and can be used immediately in amplification or stored frozen.

A number of parameters may be varied without substantially affecting the sample preparation. For example, the number of particle washing steps may be varied or the particles may be separated from the supernatant by other means (e.g., filtration, precipitation, centrifugation). The solid support may have nucleic acid capture probes affixed thereto that are complementary to the specific target sequence or any particle or solid support that non-specifically binds the target nucleic acid may be used (e.g., polycationic supports as described, for example, in U.S. Pat. No. 5,599,667). For amplification, the isolated RNA was released from the capture particles using a standard low salt elution process or amplified while retained on the particles by using primers that bind to regions of the RNA not involved in base pairing with the poly-dT or in other interactions with the solid-phase matrix. The exact volumes and proportions described above are not critical and may be varied so long as significant release of nuclear material does not occur. Vortex mixing is preferred for small-scale preparations but other mixing procedures may be substituted. It is important, however, that samples derived from biological tissue be treated to prevent coagulation and that the ionic strength of the lysing solution be at least about 150 mM, preferably 150 mM to 1 M, because lower ionic strengths lead to nuclear material contamination (e.g., DNA) that increases viscosity and may interfere with amplification and/or detection steps to produce false positives. Lithium salts are preferred in the lysing solution to prevent RNA degradation, although other soluble salts (e.g., NaCl) combined with one or more known RNase inhibitors would be equally effective.

By “solid support” is meant a material that is essentially insoluble under the solvent and temperature conditions of the method comprising free chemical groups available for joining an oligonucleotide or nucleic acid. Preferably, the solid support is covalently coupled to an oligonucleotide designed to bind, either directly or indirectly, a target nucleic acid. When the target nucleic acid is an mRNA, the oligonucleotide attached to the solid support is preferably a poly-T sequence. A preferred solid support is a particle, such as a micron- or submicron-sized bead or sphere. A variety of solid support materials are contemplated, such as, for example, silica, polyacrylate, polyacrylamide, metal, polystyrene, latex, nitrocellulose, polypropylene, nylon or combinations thereof. More preferably, the solid support is capable of being attracted to a location by means of a magnetic field, such as a solid support having a magnetite core. Particularly preferred supports are monodisperse magnetic spheres.

A number of template dependent processes are available to amplify the target sequences of interest present in a sample. One of the best known amplification methods is the polymerase chain reaction (PCR™) which is described in detail in U.S. Pat. Nos. 4,683,195, 4,683,202 and 4,800,159. Briefly, in PCR™, two primer sequences are prepared which are complementary to regions on opposite complementary strands of the target sequence. An excess of deoxynucleoside triphosphates is added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction product and the process is repeated. Preferably reverse transcription and PCR™ amplification procedure may be performed in order to quantify the amount of mRNA amplified. Polymerase chain reaction methodologies are well known in the art.

One preferred methodology for polynucleotide amplification employs RT-PCR, in which PCR is applied in conjunction with reverse transcription. Typically, RNA is extracted from a biological sample, such as blood, serum, lymph node, bone marrow, sputum, urine and tumor biopsy samples, and is reverse transcribed to produce cDNA molecules. PCR amplification using at least one specific primer generates a cDNA molecule, which may be separated and visualized using, for example, gel electrophoresis. Amplification may be performed on biological samples taken from a patient and from an individual who is not afflicted with a cancer. The amplification reaction may be performed on several dilutions of cDNA spanning two orders of magnitude. A two-fold or greater increase in expression in several dilutions of the test patient sample as compared to the same dilutions of the non-cancerous sample is typically considered positive.

Any of a variety of commercially available kits may be used to perform the amplification step. One such amplification technique is inverse PCR (see Triglia et al., Nucl. Acids Res. 16:8186, 1988), which uses restriction enzymes to generate a fragment in the known region of the gene. The fragment is then circularized by intramolecular ligation and used as a template for PCR with divergent primers derived from the known region. Within an alternative approach, sequences adjacent to a partial sequence may be retrieved by amplification with a primer to a linker sequence and a primer specific to a known region. The amplified sequences are typically subjected to a second round of amplification with the same linker primer and a second primer specific to the known region. A variation on this procedure, which employs two primers that initiate extension in opposite directions from the known sequence, is described in WIPO International Patent Application No.: WO 96/38591. Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR (Lagerstrom et al., PCR Methods Applic. 1:111-19, 1991) and walking PCR (Parker et al., Nucl. Acids. Res. 19:3055-60, 1991). Other methods employing amplification may also be employed to obtain a full length cDNA sequence.

Another method for amplification is the ligase chain reaction (referred to as LCR), disclosed in Eur. Pat. Appl. Publ. No. 320,308. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750, describes an alternative method of amplification similar to LCR for binding probe pairs to a target sequence.

Qbeta Replicase, described in PCT Intl. Pat. Appl. Publ. No. PCT/US87/00880, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[α-thio]triphosphates in one strand of a restriction site (Walker et al., 1992), may also be useful in the amplification of nucleic acids in the present invention.

Strand Displacement Amplification (SDA) is another method of carrying out isothermal amplification of nucleic acids which involves multiple rounds of strand displacement and synthesis, i.e. nick translation. A similar method, called Repair Chain Reaction (RCR) is another method of amplification which may be useful in the present invention and is involves annealing several probes throughout a region targeted for amplification, followed by a repair reaction in which only two of the four bases are present. The other two bases can be added as biotinylated derivatives for easy detection. A similar approach is used in SDA.

Sequences can also be detected using a cyclic probe reaction (CPR). In CPR, a probe having a 3′ and 5′ sequences of non-target DNA and an internal or “middle” sequence of the target protein specific RNA is hybridized to DNA which is present in a sample. Upon hybridization, the reaction is treated with RNaseH, and the products of the probe are identified as distinctive products by generating a signal that is released after digestion. The original template is annealed to another cycling probe and the reaction is repeated. Thus, CPR involves amplifying a signal generated by hybridization of a probe to a target gene specific expressed nucleic acid.

Still other amplification methods described in Great Britain Pat. Appl. No. 2 202 328, and in PCT Intl. Pat. Appl. Publ. No. PCT/US89/01025, may be used in accordance with the present invention. In the former application, “modified” primers are used in a PCR-like, template and enzyme dependent synthesis. The primers may be modified by labeling with a capture moiety (e.g., biotin) and/or a detector moiety (e.g., enzyme). In the latter application, an excess of labeled probes is added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS) (Kwoh et al, 1989; PCT Intl. Pat. Appl. Publ. No. WO 88/10315), including nucleic acid sequence based amplification (NASBA) and 3SR. In NASBA, the nucleic acids can be prepared for amplification by standard phenol/chloroform extraction, heat denaturation of a sample, treatment with lysis buffer and minispin columns for isolation of DNA and RNA or guanidinium chloride extraction of RNA. These amplification techniques involve annealing a primer that has sequences specific to the target sequence. Following polymerization, DNA/RNA hybrids are digested with RNase H while double stranded DNA molecules are heat-denatured again. In either case the single stranded DNA is made fully double stranded by addition of second target-specific primer, followed by polymerization. The double stranded DNA molecules are then multiply transcribed by a polymerase such as T7 or SP6. In an isothermal cyclic reaction, the RNAs are reverse transcribed into DNA, and transcribed once again with a polymerase such as T7 or SP6. The resulting products, whether truncated or complete, indicate target-specific sequences.

Eur. Pat. Appl. Publ. No. 329,822, disclose a nucleic acid amplification process involving cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from resulting DNA: RNA duplex by the action of ribonuclease H(RNase H, an RNase specific for RNA in a duplex with either DNA or RNA). The resultant ssDNA is a second template for a second primer, which also includes the sequences of an RNA polymerase promoter (exemplified by T7 RNA polymerase) 5′ to its homology to its template. This primer is then extended by DNA polymerase (exemplified by the large “Klenow” fragment of E. coli DNA polymerase I), resulting as a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

PCT Intl. Pat. Appl. Publ. No. WO 89/06700, disclose a nucleic acid sequence amplification scheme based on the hybridization of a promoter/primer sequence to a target single-stranded DNA (“ssDNA”) followed by transcription of many RNA copies of the sequence. This scheme is not cyclic; i.e. new templates are not produced from the resultant RNA transcripts. Other amplification methods include “RACE” (Frohman, 1990), and “one-sided PCR” (Ohara, 1989) which are well-known to those of skill in the art.

Compositions and Kits for the Detection of Cancer

The present invention further provides kits for use within any of the above diagnostic methods. Such kits typically comprise two or more components necessary for performing a diagnostic assay. Components may be compounds, reagents, containers and/or equipment. For example, one container within a kit may contain a monoclonal antibody or fragment thereof that specifically binds to a lung tumor protein. Such antibodies or fragments may be provided attached to a support material, as described above. One or more additional containers may enclose elements, such as reagents or buffers, to be used in the assay. Such kits may also, or alternatively, contain a detection reagent as described above that contains a reporter group suitable for direct or indirect detection of antibody binding.

The present invention also provides kits that are suitable for performing the detection methods of the present invention. Exemplary kits comprise oligonucleotide primer pairs each one of which specifically hybridizes to a distinct polynucleotide. Within certain embodiments, kits according to the present invention may also comprise a nucleic acid polymerase and suitable buffer. Exemplary oligonucleotide primers suitable for kits of the present invention are disclosed herein. Exemplary polynucleotides suitable for kits of the present invention are disclosed herein.

Alternatively, a kit may be designed to detect the level of mRNA encoding a lung tumor protein in a biological sample. Such kits generally comprise at least one oligonucleotide probe or primer, as described above, that hybridizes to a polynucleotide encoding a lung tumor protein. Such an oligonucleotide may be used, for example, within a PCR or hybridization assay. Additional components that may be present within such kits include a second oligonucleotide and/or a diagnostic reagent or container to facilitate the detection of a polynucleotide encoding a lung tumor protein.

In other related aspects, the present invention further provides compositions useful in the methods disclosed herein. Exemplary compositions comprise two or more oligonucleotide primer pairs each one of which specifically hybridizes to a distinct polynucleotide. Exemplary oligonucleotide primers suitable for compositions of the present invention are disclosed herein. Exemplary polynucleotides suitable for compositions of the present invention are disclosed herein.

The following Examples are offered by way of illustration and not by way of limitation.

### EXAMPLES

### Example 1

### Multiplex Detection of Lung Tumors

A Multiplex Real-time PCR assay was established in order to simultaneously detect the expression of four lung cancer-specific genes: L762 (SEQ ID NO: 1), L984 (SEQ ID NO:3), L550 (SEQ ID NO:5) and L552 (SEQ ID NO:7). In contrast to detection approaches relying on expression analysis of single lung cancer-specific genes, this Multiplex assay was able to detect all lung tumor samples tested and analyze their combined mRNA expression profile in adenocarcinoma, squamous, small cell and large cell lung tumors. L552S and L550S complement each other in detecting predominantly adenocarcinomas, L762S detects squamous cell carcinomas and L984P detects small cell carcinomas (see Table 1).

The primers and probes were designed to be intron spanning (exon specific) to eliminate any reactivity with genomic DNA making them suitable for use in blood samples without having to DNAse treat mRNA samples. They were also designed to produce amlicons of different sizes to allow gel differentiation of end products if necessary.

The assay was carried out as follows: L552S (SEQ ID NO: 7), L550 (SEQ ID NO: 5), L762 (SEQ ID NO: 1), L984 (SEQ ID NO: 3) and specific primers, and specific Taqman probes, were used to analyze their combined mRNA expression profile in lung tumors. The primers and probes are shown below:

| | |
|---|---|
| L552S: | |
| Forward Primer: | |
| 5′ GACGGCATGAGCGACACACA. | (SEQ ID NO:9) |
| Reverse Primer: | |
| 5′ CCATGTCGCGCACTGGGATC. | (SEQ ID NO:10) |
| Probe (FAM-5′-3′-TAMRA): | |
| CTGAAAGTCGGGATCCTACACCTGGGCA. | (SEQ ID NO:11) |
| L550P: | |
| Forward Primer: | |
| 5′ GGCCACCGTCTGGATTCTTC. | (SEQ ID NO:12) |
| Reverse Primer: | |
| 5′ GAAGAATCCAGACGGTGGCC. | (SEQ ID NO:13) |
| Probe (FAM-5′-3′-TAMRA): | |
| CCGCCCCAAG ATCAAATCCA CAAACC. | (SEQ ID NO:14) |
| L762S: | |
| Forward Primer: | |
| 5′ ATGGCAGAGGCTGACAGACTC. | (SEQ ID NO:15) |
| Reverse Primer: | |
| 5′ TTCAACCACCTCAAATCCTTTCTTA. | (SEQ ID NO:16) |
| Probe (FAM-5′-3′-TAMRA) | |
| TCGACAGCAAAGGAGAGATCAGAGCCC. | (SEQ ID NO:17) |
| L984P: | |
| Forward Primer: | |
| 5′ TTACGACCCGCTCAGCCC. | (SEQ ID NO:18) |
| Reverse Primer: | |
| 5′ CTCCCAACGCCACTGACAA. | (SEQ ID NO:19) |
| Probe (FAM-5′-3′-TAMRA): | |
| CCAGGCCGAGCCCCTCAGAACC. | (SEQ ID NO:20) |

The assay conditions were:

Taqman Protocol (7700 Perkin Elmer):

In 25 μl final volume: 1× Buffer A, 5 mM MgCl, 0.2 mM dCTP, 0.2 mM dATP, 0.4 mM dUTP, 0.2 mM dGTP, 0.01 U/μl AmpErase UNG, 0.0375 U/μl TaqGold, 8% (v/v) Glycerol, 0.05% (v/v) (Sigma), Gelatin, 0.05% (v/v) (Sigma), Tween20 0.1% v/v (Sigma), 300 mM of each forward and reverse primer for L762P, 50 mM of each forward and reverse primer from (L552S, L984P, L550S, L984P) 2 pmol of each gene specific Taqman probe (L552S, L550S, L984P) and template cDNA. The PCR reaction was carried out at one cycle at 95° C. for 10 minutes, followed by 50 cycles at 95° C. for 15 seconds, 60° C. for 1 minute, and 68° C. for 1 minute (ABI Prism 7900HO Sequence Detection System, Foster City, Calif.).

Since each primer set in the multiplex assay results in a band of unique length, expression signals of the four genes of interest was measured individually by agarose gel analysis. The combined expression signal of all four genes can also be measured in real-time on an ABI 7700 Prism sequence detection system (Applied Biosystems, Foster City, Calif.). Although specific primers have been described herein, different primer sequences, different primer or probe labeling and different detection systems could be used to perform this multiplex assay. For example, a second fluorogenic reporter dye could be incorporated for parallel detection of a reference gene by real-time PCR. Or, for example a SYBR Green detection system could be used instead of the Taqman probe approach. Table 2 shows the reactivity of the multiplex PCR with different lung tumor types and normal lung tissue.

TABLE 2
EXPRESSION OF LUNG CANCER MULTIPLEX
GENES (L762P, L552S, L550S, L984P)
IN LUNG TUMOR AND NORMAL LUNG

| Lung Tumor Type | Positive Samples/Samples Tested |
|---|---|
| Adenocarcinoma | 21/24 |
| Squamous | 17/18 |
| Large Cell | 5*/5 |
| Small Cell | 5/6 |
| Normal Lung Tissue | 0/12 |
| Total Tumors | 48/53 |
| % Positive Tumors | 90.57% |

Cut-off Value = Mean normal lung + 3 SD = 0.901
*One sample at cut-off

### Example 2

### Multiplex Detection of Lung Tumors

Six additional Multiplex Real-time PCR assays were established in order to simultaneously detect the expression of various combinations of recognized lung antigens: L762 (SEQ ID NO:1), L984 (SEQ ID NO:3), L550 (SEQ ID NO:5), L552 (SEQ ID NO:7), L763 (SEQ ID NO: 21) and L587 (SEQ ID NO:26). The six groups consisted of:

Group 1: L762, L552, L550 and L984

Group 2: L763, L552, L550 and L984

Group 3: L763, L552, L587 and L984

Group 4: L763, L550, L587 and L984

Group 5: L763, L550 and L587

Group 6: L762, L984, L550 and L587

The assays were carried out described above in Example 1 to analyze the combined mRNA expression profile in lung tumors. The primers and probes for L552S, L550P, L762S, L984P are as described in Example 1. primers and probes for L763 and L587 are described below:

| | |
|---|---|
| L763S: | |
| Forward Primer: | |
| 5′ ATTCCAGGCGACATCCTCACT. | (SEQ ID NO:23) |
| Reverse Primer: | |
| 5′ GTTTATCCCTGAGTCCTGTTTCCA. | (SEQ ID NO:24) |
| Probe (FAM-5′-3′-TAMRA): | |
| TGTGCACCATTGGCTTCTAGGCACTCC. | (SEQ ID NO:25) |
| L587: | |
| Forward Primer: | |
| 5′ CCCAGAGCTGTGTTAAGGGATC. | (SEQ ID NO:28) |
| Reverse Primer: | |
| 5′ GTTAAGCGGGATTTCATGTACGA. | (SEQ ID NO:29) |
| Probe (FAM-5′-3′-TAMRA): | |
| AGAACCTGAACCCGTAAAGAAGCCTCCC. | (SEQ ID NO:30) |

The lung antigens that make up the six multiplex assays are able to detect all lung tumor samples tested and were analyzed for their combined mRNA expression profile in adenocarcinoma, squamous, small cell and large cell lung tumors. The results of these assays is presented in Table 3.

TABLE 3
EXPRESSION OF LUNG CANCER MULTIPLEX
GENES IN LUNG TUMOR AND NORMAL LUNG

| | Positive Samples/Samples Tested | | | | | |
|---|---|---|---|---|---|---|
| | Group | Group | Group | Group | Group | Group |
| Lung Tumor Type | 1 | 2 | 3 | 4 | 5 | 6 |
| Adenocarcinoma | 21/24 | 21/24 | 20/24 | 22/24 | 22/24 | 22/24 |
| Squamous | 17/18 | 17/18 | 18/18 | 18/18 | 18/18 | 18/18 |
| Large Cell | 5/5 | 3/5 | 4/5 | 3/5 | 3/5 | 4/5 |
| Small Cell | 1/2 | 1/2 | 1/2 | 2/2 | 1/2 | 2/2 |
| Other | 2/2 | 2/2 | 2/2 | 2/2 | 2/2 | 2/2 |
| Normal Lung Tissue | 0/12 | 0/12 | 0/12 | 0/13 | 0/13 | 0/13 |
| Total Tumors | 46/51 | 44/51 | 45/51 | 47/51 | 46/51 | 48/51 |
| % Positive Tumors | 90.20% | 86.27% | 88.24% | 92.16% | 90.20% | 94.12% |
| | CO = 0.9 | CO = 4.7 | CO = 1.08 | CO = 1.88 | CO = 2.2 | CO = 5.5 |

Cut-off Value (CO) = Mean normal lung + 3 SD

Mulitplex assays using groups 1, 4 and 6 were next used to detect circulating tumor cells in peripheral blood samples from 17 lung cancer patients undergoing various types of treatments. In addition, a single gene assay using lung antigen L523 (SEQ ID NO:31) was carried out in parallel using the primers as described in SEQ ID NOs:33 and 34. Six normal donors were included as controls. The assays were carried out as described above in Example 1. The cut off value for detection in the assay being the mean of the normal lung samples +3 standard deviations.

Group 1 antigens were detected in 5/17 samples tested. Group 4 antigens were detected in 4/17 samples and Group 6 antigens were detected in 8/17 samples. L523 was detected as a single gene in 7/17 samples tested. The combination of antigens in Group 6 was the most sensitive for lung tumor detection in tissue and blood of the groups tested.

### Example 3

### Further Characterization of Multiplex Detection Assay

A. Materials and Methods

1. Tissue Sources and RNA Extraction

Primary cancer tissues and healthy tissues were obtained from Cooperative Human Tissue Network (CHTN), National Disease Research Interchange (NDRI), and other clinical sources. SCLC tumor cell lines were obtained from American Type Culture Collection (ATCC). Total RNA was isolated from homogenized tissue samples using TRIZOL® reagant (Invitrogen #15596-018). DNase treatment was performed using DNase I (Ambion #2222) followed by phenol/chloroform extraction and ethanol precipitation.

2. Blood Sources and RNA Extraction

Ten milliliters peripheral blood samples were drawn from cancer patients into EDTA containing vacutainers at Swedish Medical Oncology Clinic, Seattle, Wash., and additional peripheral blood samples were obtained from ProteoGenex, Manhattan Beach, Calif. Samples were processed within three hours using RosetteSep™ tumor cell enrichment antibody cocktail (StemCell Technologies Inc. #15122). The enrichment cocktail contains tetrameric antibodies that cross-link normal hematopoietic cells (CD45), granulocytes (CD66b) and monocytes (CD36) to red blood cells (glycophorin A). Depleted tumor mononuclear cells were collected using ACCUSPIN™ System-HISTOPAQUE®-1077 (Sigma-Aldrich #A-6929). mRNA was isolated using the Roche mRNA Isolation Kit (#1741985). DNase treatment was performed using DNA-free™ (Ambion #1906) according to the manufacturer's protocol.

3. cDNA Synthesis

mRNA was reverse transcribed into cDNA using Oligo(dT)₂₀ Primer and SuperScript™ II Reverse Transcriptase (Invitrogen #18418-020 and #18064-014) for 1 hour at 42° C.

4. Primers, Probes, and Real-Time PCR

Specific primers and 6-carboxy-fluorescein (FAM)-labeled TaqMan® probes were used in combination to detect mRNA expression of different cancer-specific genes simultaneously. The primers were designed to be intron-spanning and exon-specific to eliminate reactivity with genomic DNA. In addition, the multigene RT-PCR test was engineered such that unique amplicon sizes resulted from amplification of individual target genes, which could be used to indicate tumor type. Typical 25 μl PCR reaction mixtures included 1× TaqMan® Buffer (Applied Biosystems, TaqMan PCR Core Reagents Kit #4304439), 5 mM MgCl₂, 0.2 mM dCTP, 0.2 mM dATP, 0.2 mM dUTP, 0.2 mM dGTP, 0.01 U/μl AmpErase® UNG, 0.0375 U/μl AmpliTaq Gold® DNA Polymerase, 8% v/v glycerol, 0.05% v/v gelatin, 0.01% v/v Tween20, 300 nM L762-2ISF (SEQ ID NO:15) 5′-ATGGCAGAGGCTGACAGACTC-3′ and L762-2ISR (SEQ ID NO:16) 5′-TTCAACCACCTCAAATCCTTTCTTA-3′ primers; 200 nM L587-2ISF (SEQ ID NO:28) 5′-CCCAGAGCTGTGTTAAGGGATC-3′ and L587-2ISR (SEQ ID NO:29) 5′-GTTAAGCGGGATTTCATGTACGA-3′ primers; and 50 nM each of L550-2ISF (SEQ ID NO:12) 5′-GGCCACCGTCTGGATTCTTC-3′, L550-2ISR (SEQ ID NO:35) 5′-TCGACTTATAGTCAGCAACATCCTTCT-3′, L984-1ISF (SEQ ID NO:18) 5′-TTACGACCCGCTCAGCCC-3′, and L984-1R (SEQ ID NO:19) 5′-CTCCCAACGCCACTGACAA-3′ primers. Each reaction also contained 3 pmol of each gene specific probe: L762 5′-6FAM-TCGACAGCAAAGGAGAGATCAGAGCCC-3′-TAMRA (SEQ ID NO:17); L587 5′-6FAM-AGAACCTGAACCCGTAAAGAAGCCTCCC-3′-TAMRA (SEQ ID NO:30); L550 5′-6FAM-CCGCCCCAAGATCAAATCCACAAACC-3′-TAMRA (SEQ ID NO:14); and L984 5′-6FAM-CCAGGCCGAGCCCCTCAGAACC-3′-TAMRA (SEQ ID NO:20).

The combined gene expression levels were measured by quantitative real-time PCR using the ABI PRISM® 7700 Sequence Detection System (Applied Biosystems, Foster City, Calif.) using the cycle 50° C.-2 min; 95° C.-10 min; 50 cycles of 95° C.-15 sec, 60° C.-1 min, 68° C.-1 min. Multigene (MPLX) copy numbers and actin message concentration were calculated by constructing standard curves using the TaqMan® SDS analysis software from serial dilutions of four combined purified PCR amplicons containing target gene cDNA sequences and human genomic DNA, respectively. Final MPLX copy numbers were determined as medians of triplicate reactions for blood samples and duplicate reactions for tissue samples. Actin expression was measured in separate reactions as a quality control for blood and tissue cDNA samples using reactions with 300 nM ActinF 5′-ACTGGAACGGTGAAGGTGACA (SEQ ID NO:36) and ActinR 5′-CGGCCACATTGTGAACTTTG (SEQ ID NO:37) primers, and 3 pmol actin-probe 5′-6FAM-CAGTCGGTTGGAGCGAGCATCCC-3′-TAMRA (SEQ ID NO:38). The expression levels for tissue samples are reported as MPLX copies normalized per 1000 pg of actin. Blood samples with actin expression <50 pg were excluded from analysis.

5. Electrophoresis

PCR products were analyzed by agarose gel electrophoresis to determine differential expression of the four-gene multiplex including L762P (249 bp), L550S (204 bp), L587S (169 bp), and L984P (157 bp). Four percent agarose E-Gels® (Invitrogen #G5018-04) were used to separate products at 70V for 30 minutes according to the manufacturer's recommendations.

B. Results

As noted in Example 2, a panel of 51 lung tumors and 13 normal lung tissues was evaluated by a four-gene multiplex RT-PCR assay to determine specificity of the test for lung cancer. Based on a cut-off value of 3 standard deviations above the mean MPLX copies of the 13 normal lung tissues, MPLX sensitivity for lung cancers of greater than 94% was achieved (Table 3; group 6). High MPLX expression signal was detected in 2/2 bronchoalveolar/neuroendocrine tumors, 2/2 SCLC primary tumors (and 2/2 SCLC cell lines), 4/5 large cell carcinomas, 18/18 squamous cell carcinomas, and 22/24 adenocarcinomas in comparison to normal lung tissues (Table 3, group 6).

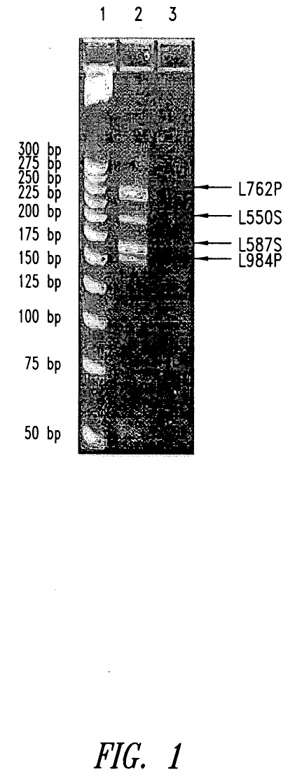

PCR products were separated by agarose gel electrophoresis to determine which genes were expressed in individual tumors (FIG. 1). L762P expression is relatively restricted to squamous cell carcinomas (Table 4). L550S and L587S expression is more promiscuous, but these markers most often appear as complementary expressed genes in adenocarcinomas (Table 4). L984P is present in most SCLC but is rare in NSCLC (Table 4). Interestingly, in some cases, all four lung markers were amplified from a single sample.

TABLE 4
Genes Selected for Lung Cancer Multiplex Real-Time RT-PCR

| Gene | | |
|---|---|---|
| Name | Gene Identity | Tumor Specificity |
| L550S | human high mobility group | Adenocarcinoma, |
| | protein 2a (HMG2a) | complimentary |
| | | expression to L587S |
| L587S | novel | Adenocarcinoma, |
| | | complimentary |
| | | expression to L550S |
| L762P | calcium activated chloride | Squamous and Large Cell |
| | channel 2 (CLCA2) | Lung Carcinoma |
| L984P | novel; partial homology | Small Cell Lung |
| | with Aschaetescute | Carcinoma |
| | homologous protein (ASH1) | |

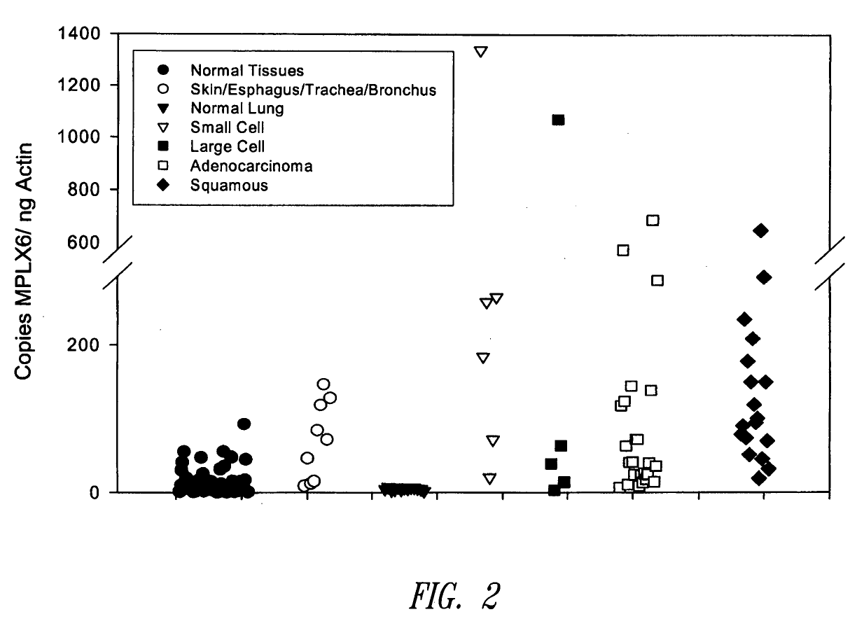

In order to examine the specificity of the assay relative to normal tissues, a panel of 160 human tissues and cell lines was evaluated by the four-gene multiplex RT-PCR assay. Low reactivity was found in 85 normal tissues, while a slightly elevated MPLX signal was detected in normal skin, esophagus, trachea, and bronchus (FIG. 2). No significant MPLX signal was found in activated and resting peripheral blood samples (n=7), with mean expression of 3.33+/−3.86 MPLX copies. Additionally, we evaluated a panel of 100 tumor tissues representing 12 tumor types by the four-gene multiplex RT-PCR assay and found at least three-fold elevated MPLX expression, relative to the average MPLX signal observed for normal tissues of the same type as the tumors being tested, in 2/6 pancreatic cancers, 1/6 kidney tumors, 5/6 bladder tumors, 2/6 breast tumors 5/6 ovarian tumors and 2/2 stomach tumors.

Given the low background detection of the four cancer-associated markers in peripheral blood, we evaluated 108 blood samples from 49 lung cancer patients at various stages of disease and undergoing different treatment regimens. Each of the four major tumor types was represented (Table 5). Repeat draws were obtained from 20 of the patients to monitor disease progression during therapy and potential relapse. The patient population included 24 males, 21 females, and 4 donors that did not provide gender information. The mean age of the patients was 61.8 years.

TABLE 5
Patient Demographics

| | |
|---|---|
| Age Distribution (Mean) | 61.8 years (41-82) |
| Gender | 24 male, 21 female, 4 no information |

| | Number of | Total | | |
|---|---|---|---|---|
| | Patients | Number of | Number of | |
| | per Tumor | Draws, All | Positive | |
| Tumor Type: | Type | Patients | Draws (%) | |
| Squamous | 8 | 9 | 6 | (66.7%) |
| Adenocarcinoma | 11 | 17 | 10 | (58.8%) |
| Small Cell | 10 | 20 | 13 | (65.0%) |
| Large Cell | 1 | 4 | 2 | (50%) |
| NSCLC | 14 | 46 | 16 | (34.8%) |
| Bronchoalveolar | 4 | 11 | 2 | (18.2%) |
| Mesothelioma | 1 | 1 | 0 | (0%) |
| Total | 49 | 108 | 49 | (45.4%) |

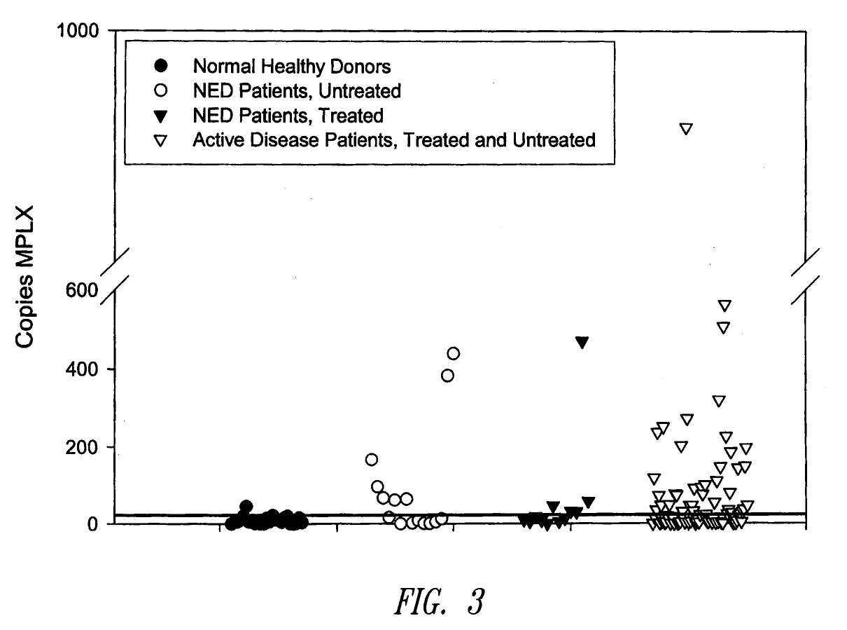

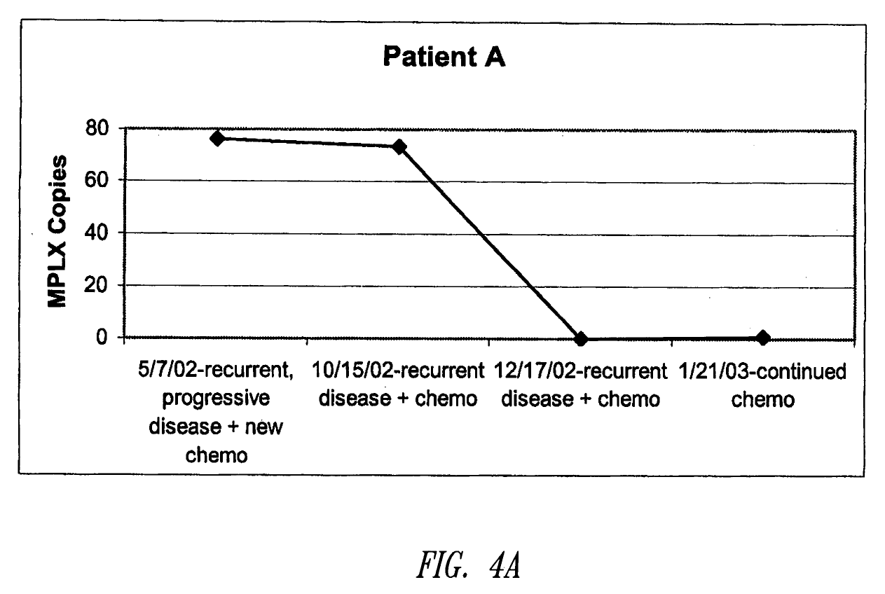

Twenty-five additional blood samples were collected from normal, healthy donors and used to establish cut-off values for positive multiplex gene expression. The cut-off value was established at 2 standard deviations above the mean of normal donor MPLX signal for blood assays (FIG. 3). Patients with no current evidence of disease (NED) were separated into groups: those receiving treatment and those not receiving treatment. 7/15 patients with no current evidence of disease receiving treatment showed MPLX copies above the cut-off value, while 5/12 patients with no current evidence of disease not receiving treatment showed MPLX copies above the cut-off value (FIG. 3). Similarly, patients with active disease were separated into groups: those receiving treatment and those not receiving treatment. 27/64 patients with active disease receiving treatment had significant MPLX signal, while 10/17 patients with active disease not receiving treatment also had significant MPLX signal (FIG. 3). Multigene signals for the positive patients ranged from 27.80 MPLX copies to 991.74 MPLX copies.

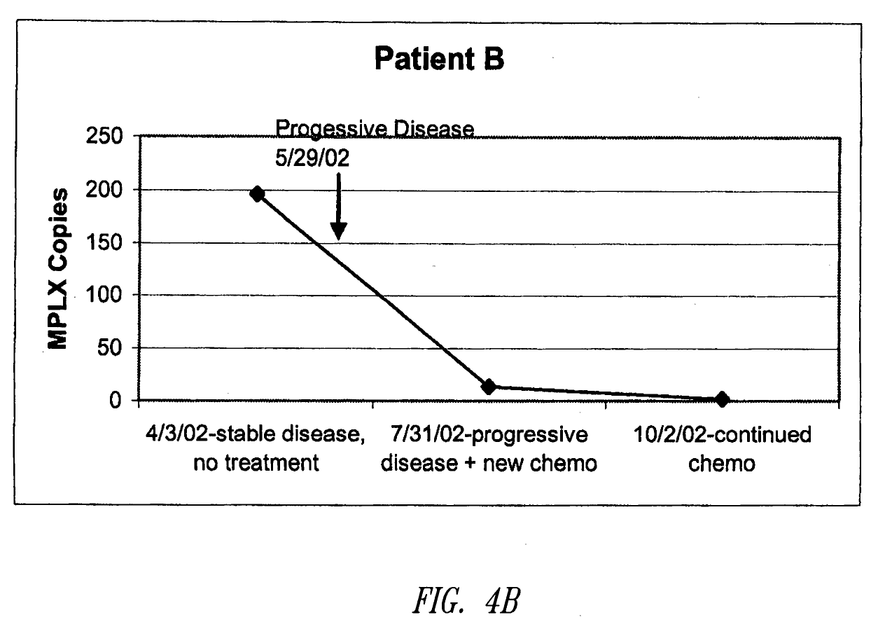

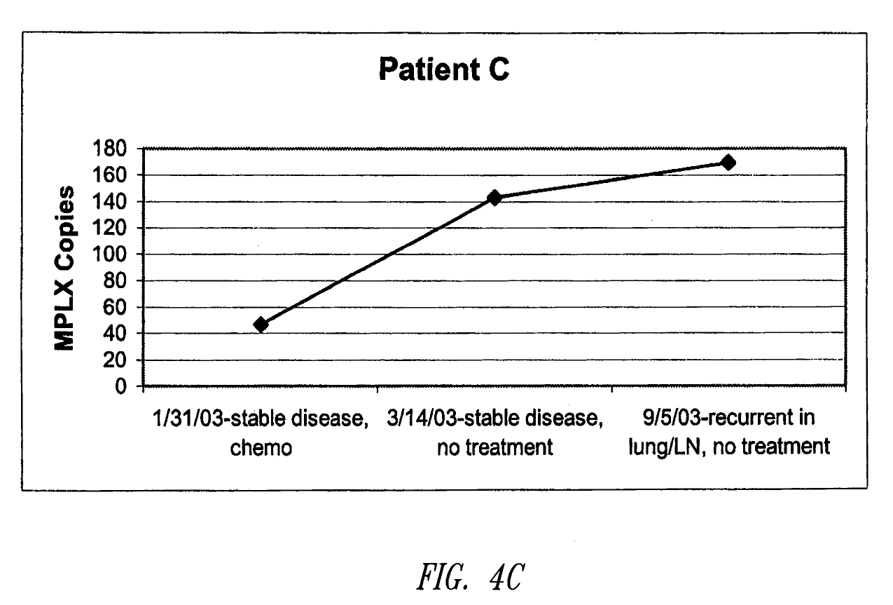

Repeat draws from individual patients were monitored for changes in gene expression related to treatment regimens, relapse or changes in disease state. Patient A with large cell lung carcinoma in a progressive disease state was treated with a new course of chemotherapy. Over a 7-month treatment session, MPLX copy number fell below the cut-off value, suggesting a reduction in circulating tumor cells in Patient A's blood (FIG. 4A). Similar results were seen in Patient B with small cell lung carcinoma, who received a new chemotherapy treatment at onset of progressive disease. Patient B experienced a pronounced drop in MPLX copies within a 6-month period (FIG. 4B). In Patient C with squamous cell carcinoma, elevated MPLX copies in two blood samples were detected 6 months prior to clinical diagnosis of recurrent cancer in the lung with lymph node involvement (FIG. 4C).

From the foregoing it will be appreciated that, although specific embodiments of the invention have been described herein for purposes of illustration, various modifications may be made without deviating from the spirit and scope of the invention. Accordingly, the invention is not limited except as by the appended claims.

We claim:

1. A method for detecting the presence of cancer cells in a biological sample comprising the steps of:
(a) detecting the level of expression in the biological sample of at least two cancer-associated markers selected from the group consisting of L762P, L550S, L587S and L984P; and
(b) comparing the level of expression detected in the biological sample for each marker to a predetermined cut-off value for each marker;
wherein a detected level of expression above the predetermined cut-off value for one or more markers is indicative of the presence of cancer cells in the biological sample.

2. The method of claim 1, wherein step (a) comprises detecting the level of mRNA expression.

3. The method of claim 2, wherein step (a) comprises detecting the level of mRNA expression using a nucleic acid hybridization technique.

4. The method of claim 2, wherein step (a) comprises detecting the level of mRNA expression using a nucleic acid amplification method.

5. The method of claim 4, wherein step (a) comprises detecting the level of mRNA expression using a nucleic acid amplification method selected from the group consisting of transcription-mediated amplification (TMA), polymerase chain reaction amplification (PCR), ligase chain reaction amplification (LCR), strand displacement amplification (SDA), and nucleic acid sequence based amplification (NASBA).

6. The method of claim 2, wherein the L762P mRNA comprises a nucleic acid sequence set forth in SEQ ID NO: 1 or a nucleic acid sequence encoding an amino acid sequence set forth in SEQ ID NO: 2.

7. The method of claim 2, wherein the L550S mRNA comprises a nucleic acid sequence set forth in SEQ ID NO: 5.

8. The method of claim 2, wherein the L587S mRNA comprises a nucleic acid sequence set forth in SEQ ID NO: 26 or a nucleic acid sequence encoding an amino acid sequence set forth in SEQ ID NO: 27.

9. The method of claim 2, wherein the L984P mRNA comprises a nucleic acid sequence set forth in SEQ ID NO: 3 or 39 or a nucleic acid sequence encoding an amino acid sequence set forth in SEQ ID NO: 4 or 40.

10. The method of claim 1, wherein step (a) comprises detecting the level of protein expression.

11. The method of claim 10, wherein step (a) comprises detecting the level of protein expression using an immunoassay.

12. The method of claim 11, wherein step (a) comprises detecting the level of protein expression using an immunoassay selected from the group consisting of an ELISA, an immunohistochemical assay, an immunocytochemical assay, and a flow cytometry assay of antibody-labeled cells.

13. The method of claim 10, wherein the L762P protein comprises an amino acid sequence set forth in SEQ ID NO: 2.

14. The method of claim 10, wherein the L587S protein comprises an amino acid sequence set forth in SEQ ID NO: 27.

15. The method of claim 10, wherein the L984P protein comprises an amino acid sequence set forth in SEQ ID NO: 4 or 40.

16. The method of claim 1, wherein the cancer is a lung cancer, pancreatic cancer, kidney cancer, bladder cancer or breast cancer.

17. The method of claim 16, wherein the cancer is a lung cancer.

18. The method of claim 17, wherein the cancer is a small cell lung cancer or a non-small cell lung cancer.

19. The method of claim 1, wherein the biological sample is a sample suspected of containing cancer-associated markers, antibodies to such cancer-associated markers or cancer cells expressing such markers or antibodies.

20. The method of claim 19, wherein the biological sample is selected from the group consisting of a biopsy sample, lavage sample, sputum sample, serum sample, peripheral blood sample, lymph node sample, bone marrow sample, urine sample, and pleural effusion sample.

21. A method for detecting the presence of lung cancer cells in a biological sample comprising the steps of:
(a) detecting the level of mRNA expression in the biological sample of at least two cancer-associated markers selected from the group consisting of L762P, L550S, L587S and L984P, using a nucleic acid amplification method; and
(b) comparing the level of expression detected in the biological sample for each marker to a predetermined cut-off value for each marker;
wherein a detected level of expression above the predetermined cut-off value for one or more markers is indicative of the presence of lung cancer cells in the biological sample.

22. The method of claim 21, wherein the nucleic acid amplification method is selected from the group consisting of transcription-mediated amplification (TMA), polymerase chain reaction amplification (PCR), ligase chain reaction amplification (LCR), strand displacement amplification (SDA), and nucleic acid sequence based amplification (NASBA).

23. The method of claim 21, wherein the L762P mRNA comprises a nucleic acid sequence set forth in SEQ ID NO: 1 or a nucleic acid sequence encoding an amino acid sequence set forth in SEQ ID NO: 2.

24. The method of claim 21, wherein the L550S mRNA comprises a nucleic acid sequence set forth in SEQ ID NO: 5.

25. The method of claim 21, wherein the L587S mRNA comprises a nucleic acid sequence set forth in SEQ ID NO: 26 or a nucleic acid sequence encoding an amino acid sequence set forth in SEQ ID NO:27.

26. The method of claim 21, wherein the L984P mRNA comprises a nucleic acid sequence set forth in SEQ ID NO: 3 or 39 or a nucleic acid sequence encoding an amino acid sequence set forth in SEQ ID NO: 4 or 40.

27. The method of claim 21, wherein the cancer is a small cell lung cancer or a non-small cell lung cancer.

28. A method for detecting the presence of lung cancer cells in a biological sample comprising the steps of:
(a) detecting the level of protein expression in the biological sample of at least two cancer-associated markers selected from the group consisting of L762P, L550S, L587S and L984P, using an immunoassay; and
(b) comparing the level of expression detected in the biological sample for each marker to a predetermined cut-off value for each marker;
wherein a detected level of expression above the predetermined cut-off value for one or more markers is indicative of the presence of lung cancer cells in the biological sample.

29. The method of claim 28, wherein the immunoassay is selected from the group consisting of an ELISA, an immunohistochemical assay, an immunocytochemical assay, and a flow cytometry assay of antibody-labeled cells.

30. The method of claim 28, wherein the L762P protein comprises an amino acid sequence set forth in SEQ ID NO: 2.

31. The method of claim 28, wherein the L587S protein comprises an amino acid sequence set forth in SEQ ID NO: 27.

32. The method of claim 28, wherein the L984P protein comprises an amino acid sequence set forth in SEQ ID NO: 4 or 40.

33. The method of claim 28, wherein the cancer is a small cell lung cancer or a non-small cell lung cancer.

34. A composition for detecting cancer cells in a biological sample comprising at least two of:
a) a first oligonucleotide specific for L762P;
b) a second oligonucleotide specific for L550S;
c) a third oligonucleotide specific for L587S; and
d) a fourth oligonucleotide specific for L984P.

35. The composition of claim 34, wherein the first oligonucleotide is specific for an L762P nucleic acid sequence set forth in SEQ ID NO: 1 or a nucleic acid sequence encoding an amino acid sequence set forth in SEQ ID NO: 2, the second oligonucleotide is specific for an L550S nucleic acid sequence set forth in SEQ ID NO:5, the third oligonucleotide is specific for an L587S nucleic acid sequence set forth in SEQ ID NO: 26 or a nucleic acid sequence encoding an amino acid sequence set forth in SEQ ID NO:27, and the fourth oligonucleotide is specific for an L984P nucleic acid sequence set forth in SEQ ID NO: 3 or 39 or a nucleic acid sequence encoding an amino acid sequence set forth in SEQ ID NO: 4 or 40.

36. A diagnostic kit for detecting cancer cells in a biological sample comprising at least two of:
a) a first oligonucleotide specific for L762P;
b) a second oligonucleotide specific for L550S;
c) a third oligonucleotide specific for L587S; and
d) a fourth oligonucleotide specific for L984P.

37. The kit of claim 36, wherein the first oligonucleotide is specific for an L762P nucleic acid sequence set forth in SEQ ID NO: 1 or a nucleic acid sequence encoding an amino acid sequence set forth in SEQ ID NO: 2, the second oligonucleotide is specific for an L550S nucleic acid sequence set forth in SEQ ID NO:5, the third oligonucleotide is specific for an L587S nucleic acid sequence set forth in SEQ ID NO: 26 or a nucleic acid sequence encoding an amino acid sequence set forth in SEQ ID NO: 27, and the fourth oligonucleotide is specific for an L984P nucleic acid sequence set forth in SEQ ID NO: 3 or 39 or a nucleic acid sequence encoding an amino acid sequence set forth in SEQ ID NO: 4 or 40.

38. A composition for detecting cancer cells in a biological sample comprising at least two of:
a) a first primer pair specific for L762P;
b) a second primer pair specific for L550S;
c) a third primer pair specific for L587S; and
d) a fourth primer pair specific for L984P.

39. The composition of claim 38, wherein the first, second, third and fourth primer pairs are effective in a nucleic acid amplification method for amplifying all or a portion of an L762P nucleic acid sequence set forth in SEQ ID NO: 1 or a nucleic acid sequence encoding an amino acid sequence set forth in SEQ ID NO: 2, an L550S nucleic acid sequence set forth in SEQ ID NO:5, an L587S nucleic acid sequence set forth in SEQ ID NO: 26 or a nucleic acid sequence encoding an amino acid sequence set forth in SEQ ID NO: 27, and an L984P nucleic acid sequence set forth in SEQ ID NO: 3 or 39 or a nucleic acid sequence encoding an amino acid sequence set forth in SEQ ID NO: 4 or 40, respectively.

40. A diagnostic kit for detecting cancer cells in a biological sample comprising at least two of:
a) a first primer pair specific for L762P;
b) a second primer pair specific for L550S;
c) a third primer pair specific for L587S; and
d) a fourth primer pair specific for L984P.

41. The kit of claim 40, wherein the first, second, third and fourth primer pairs are effective in a nucleic acid amplification method for amplifying all or a portion of an L762P nucleic acid sequence set forth in SEQ ID NO: 1 or a nucleic acid sequence encoding an amino acid sequence set forth in SEQ ID NO: 2, an L550S nucleic acid sequence set forth in SEQ ID NO:5, an L587S nucleic acid sequence set forth in SEQ ID NO: 26 or a nucleic acid sequence encoding an amino acid sequence set forth in SEQ ID NO:27, and an L984P nucleic acid sequence set forth in SEQ ID NO: 3 or 39 or a nucleic acid sequence encoding an amino acid sequence set forth in SEQ ID NO: 4 or 40, respectively.

42. A composition for detecting cancer cells in a biological sample comprising at least two of:
a) a first antibody specific for an L762P protein;
b) a second antibody specific for an L550S protein;
c) a third antibody specific for an L587S protein; and
d) a fourth antibody specific for an L984P protein.

43. The composition of claim 42, wherein the L762P protein comprises an amino acid sequence set forth in SEQ ID NO: 2, the L550S protein comprises an amino acid sequence set forth in SEQ ID NO: 6, the L587S protein comrpises an amino acid sequence set forth in SEQ ID NO: 27, and the L984P protein comprises an amino acid sequence set forth in SEQ ID NO: 4 or 40.

44. A diagnostic kit for detecting cancer cells in a biological sample comprising at least two of:
a) a first antibody specific for an L762P protein;
b) a second antibody specific for an L550S protein;
c) a third antibody specific for an L587S protein; and
d) a fourth antibody specific for an L984P protein.

45. The kit of claim 44, wherein the L762P protein comprises an amino acid sequence set forth in SEQ ID NO: 2, the L550S protein comprises an amino acid sequence set forth in SEQ ID NO: 6, the L587S protein comprises an amino acid sequence set forth in SEQ ID NO: 27, and the L984P protein comprises an amino acid sequence set forth in SEQ ID NO: 4 or 40.

46. An array comprising at least two of:
a) a first oligonucleotide specific for L762P;
b) a second oligonucleotide specific for L550S;
c) a third oligonucleotide specific for L587S; and
d) a fourth oligonucleotide specific for L984P.

47. The array of claim 46, wherein the first oligonucleotide is specific for an L762P nucleic acid sequence set forth in SEQ ID NO: 1 or a nucleic acid sequence encoding an amino acid sequence set forth in SEQ ID NO: 2, the second oligonucleotide is specific for an L550S nucleic acid sequence set forth in SEQ ID NO:5, the third oligonucleotide is specific for an L587S nucleic acid sequence set forth in SEQ ID NO: 26 or a nucleic acid sequence encoding an amino acid sequence set forth in SEQ ID NO: 27, and the fourth oligonucleotide is specific for an L984P nucleic acid sequence set forth in SEQ ID NO: 3 or 39 or a nucleic acid sequence encoding an amino acid sequence set forth in SEQ ID NO: 4 or 40.

48. An array comprising at least two of:
a) a first antibody specific for an L762P protein;
b) a second antibody specific for an L550S protein;
c) a third antibody specific for an L587S protein; and
d) a fourth antibody specific for an L984P protein.

49. The array of claim 48, wherein the L762P protein comprises an amino acid sequence set forth in SEQ ID NO: 2, the L550S protein comprises an amino acid sequence set forth in SEQ ID NO: 6, the L587S protein comprises an amino acid sequence set forth in SEQ ID NO: 27, and the L984P protein comprises an amino acid sequence set forth in SEQ ID NO: 4 or 40.

50. An array comprising at least two of:
a) an L762P protein or portion thereof;
b) an L550S protein or portion thereof;
c) an L587S protein or portion thereof; and
d) an L984P protein or portion thereof.

51. The array of claim 50, wherein the L762P protein comprises an amino acid sequence set forth in SEQ ID NO: 2, the L550S protein comprises an amino acid sequence set forth in SEQ ID NO: 6, the L587S protein comprises an amino acid sequence set forth in SEQ ID NO: 27, and the L984P protein comprises an amino acid sequence set forth in SEQ ID NO: 4 or 40.

52. The method of claim 10, wherein the L550S protein comprises an amino acid sequence set forth in SEQ ID NO: 6.

53. The method of claim 28, wherein the L550S protein comprises an amino acid sequence set forth in SEQ ID NO: 6.
